(19) United States
(12) Patent Application Publication
(10) Pub. No.: US 2021/0355526 A1
(43) Pub. Date: Nov. 18, 2021

(54) MOLECULAR TYPING OF MICROBES

(71) Applicant: MINISTRY OF HEALTH, STATE OF ISRAEL, Jerusalem (IL)

(72) Inventors: Israel NISSAN, Modiin (IL); Paul FREIDLIN, Rishon LeZion (IL); Drora GOLDBLATT, Tel Aviv (IL); Hasia KADAIR-SHWARTZ, Shoham (IL); Zeev DVEYRIN, Ness Ziona (IL); Rona GROSSMAN, Jerusalem (IL); Efrat RORMAN, Zur Hadassah (IL); Mor RUBINSTEIN, Ness Ziona (IL); Ephraim FASS, Rehovot (IL)

(21) Appl. No.: 17/266,639

(22) PCT Filed: Aug. 8, 2019
(86) PCT No.: PCT/IL2019/050902

§ 371 Date: Feb. 8, 2021

Related U.S. Application Data
(60) Provisional application No. 62/715,813, filed on Aug. 8, 2018.

(51) Int. Cl. C12Q 1/689; C12Q 1/686; C12Q 1/6869

(52) U.S. Cl. CPC C12Q 1/689; C12Q 1/6869; C12Q 1/686

(57) ABSTRACT

A method for characterizing spacer regions in a CRISPR array from each of a plurality of microbial DNA isolates, the method comprising: in a separate reaction well for each of the plurality of microbial DNA isolates, performing a PCR with a microbial DNA isolate and at least one pair of primers configured to amplify spacers within a CRISPR array comprised in the microbial DNA isolate and to add at least one barcode that uniquely indexes the PCR products produced in the reaction well, pooling the PCR products produced from each of the plurality of microbial DNA isolates; and sequencing the pooled PCR products with a Next Generation Sequencing (NGS) system to obtain an aggregated sequence data.

### RELATED APPLICATIONS

The present application claims benefit under 35 U.S.C. 119(e) of U.S. Provisional Applications 62/715,813 filed on Aug. 8, 2018, the disclosure of which is incorporated herein by reference.

### INTRODUCTION

Bacterial-typing refers to identification and characterization of bacterial strains. There are several different ways to carry out bacterial typing and the development of these methods has improved the ability to discriminate between bacterial strains from the same species and identify, for example, the presence of clinically relevant genes, that may be relevant to virulence or antibiotic resistance in a given bacterial species. Bacterial typing has enhanced efforts to control nosocomial infections and understand the transmission, pathogenesis and phylogeny of bacteria. For example, such a system may be used for infection control in a hospital setting, for epidemiological investigation in public health settings and to propose better treatment plans for patients. Bacterial typing has also been shown to be of importance in veterinary medicine, agriculture, research, quality control of industrial bacterial cultures and in settings where there is a need for a rapid and accurate method for pathogen subtyping and/or identification of genes associated with virulence or antibiotic resistance. Traditional bacterial typing methods have been based on phenotype, and included typing methods based on serotype or biotype, phage typing, or antibiograms. Other techniques such as pulsed-field gel electrophoresis (PFGE); multi-locus sequence typing (MLST), and sequencing of the entire bacterial genome have also been developed.

Clustered Regularly Interspaced Short Palindromic Repeats (CRISPR) together with CRISPR associated genes (Cas genes) are referred to as CRISPR/Cas systems or simply as “CRISPR systems”. A CRISPR/Cas system is known to function as a prokaryotic immune system that confers adaptive resistance as a form of acquired immunity to foreign genetic elements such as plasmids and phages. CRISPRs have been found in about 45% of sequenced bacterial genomes and 87% of sequenced archaea. Proteins encoded by the Cas genes, referred to as Cas proteins, splice relatively short DNA fragments from the foreign genetic elements into contiguous stretches of DNA in the CRISPR system known as CRISPR arrays. CRISPR arrays comprise, in alternating order, Direct Repeats (DRs) characterized by repeating sequences of microbial genomic DNA, and spacer segments (alternatively “spacers”) that are remnants of DNA fragments from past invaders of the microbe (or one of its progenitors), which was spliced into the CRISPR array. Differences in the history of previous exposure to foreign DNA results in differences in the set of spacers present in CRSIPR arrays between different microbial species as well as between individual strains in a given species.

Techniques for microbial typing, for bacteria as well as species of archaea, based on characterizing spacers in CRISPR arrays, which may be referred to herein as “CRISPR-typing”, have been developed over the past 20 years. An early use of CRISPR-typing was spacer-oligonucleotide typing, or “spoligotyping” of Mycobacterium tuberculosis (M. tuberculosis) strains. See, for example, Kamerbeek J, Schouls L, Kolk A, et al. Simultaneous detection and strain differentiation of M. tuberculosis for diagnosis and epidemiology. J Clin Microbiol. 1997;35(4):907-914. The principle of spoligotyping is PCR amplification of the CRISPR array with labeled primers that recognize the DR sequences, followed by hybridization of the PCR products to a membrane that contains probes bearing oligonucleotide DNA sequences of known spacers. However, spoligotyping is a relatively low-throughput, time-consuming process, typically taking about three days even when performed by a highly trained technician, with the number of samples that can be processed in parallel typically being limited to about 35 samples. In addition, extracting data from the film is a manual process, which is a subjective process and prone to error. Moreover, this method can only detect the presence or absence of known spacers for which a hybridization sequence is already available, and thus cannot be used as a way to detect and characterize previously uncharacterized spacers. Despite these limitations, the above-described hybridization-based spoligotyping is considered to be the “gold standard” for CRISPR-typing M. tuberculosis.

Other methods for CRISPR-typing have also been subsequently developed using, for example, spacer oligonucleotide-conjugated microspheres in liquid phase (for example Microplex from Luminex); matrix-assisted laser desorption ionization-time of flight mass spectrometry (MALDI-TOF MS), whole-genome sequencing, and DNA microarrays (for example ArrayStrip platform from Alere Technologies GmbH, Jena, Germany). However, these methods require specialized instruments and reagents that are relatively expensive, thus taking them out of reach for most labs as well as rendering them impractical as clinical or commercial assays. In addition, with the theoretical exception of whole-genome sequencing, which is particularly time-consuming and expensive, the other methods are, as already noted with respect to the gold-standard spoligotyping method, limited to detection of known spacers and are thus unsuitable for discovering new spacers or characterizing spacers of previously uncharacterized CRISPR arrays or microbial species.

### SUMMARY

An aspect of the disclosure relates to providing a relatively simple, fast, and high-throughput method for molecular typing of microbes.

In an embodiment of the disclosure, for each of the plurality of microbial DNA isolates, portions of a CRISPR array comprising one or more spacers are amplified using a polymerase chain reaction (PCR), and the PCR products are sequenced by next generation sequencing (NGS), by way of example with a 454® sequencing system (Roche®) or a MiSeq® system (Illumina®), to identify individual spacers found in the CRISPR array. NGS may alternatively be referred to in the art as massively parallel sequencing (MPS). For convenience of presentation, a method of molecular typing in accordance with an embodiment of the disclosure may be referred to as a “HiCRISPR method”.

In an embodiment of the disclosure, a HiCRISPR method comprises performing PCR amplification of DNA isolates of a plurality of microbial samples wherein each reaction well of the plurality of reactions wells contains: a microbial DNA isolate; primers, in accordance with an embodiment, that include a targeting region complementary to portions of direct repeat (DR) regions within a CRISPR array of the microbe and a unique DNA sequence, referred as a “barcode”, that indexes the primers. The products of the reactions are then pooled, and the pooled PCR products are sequenced by NGS. The pooling of PCR products from a plurality of PCR's, optionally performed in parallel, may be referred herein as “sample multiplexing”.

In an embodiment of the disclosure, primers loaded in a given reaction well comprise a same barcode, so that each of the different reaction wells (and the PCR products produced therein) is characterized by a different barcode. Alternatively, forward and reverse primers loaded in a given reaction well comprise a different barcode, such that each reaction well is characterized with a unique combination of two barcodes. In an embodiment of the disclosure, NGS sequence data produced by NGS are analyzed to demultiplex the sequence data according to the barcodes so that sequence data portions sharing a same barcode or barcode pair, and thus generated in the same reaction well from the same microbial DNA sample and the same primer pairs, are sorted together. Optionally, the sorted sequence portions are further analyzed to identify sequences encoding spacers detected by PCR from a given bacterial sample to create a spacer profile, which may be referred to as a “SPACERome”, characterizing the given microbial sample.

Optionally, a reaction well further comprises at least one additional pair of primers, each primer comprising a barcode associated with the reaction well and a targeting region comprising a sequence complementary to a gene associated with a lineage marker, antibiotic resistance or virulence.

An aspect of the disclosure relates to providing primers, which may be referred to herein as “HiCRISPR primers” that are used for a HiCRISPR process. In an embodiment of the disclosure, a HiCRISPR primer comprises a barcode and a targeting sequence that is complementary to a portion of a DR of a CRISPR array.

An aspect of the disclosure relates to providing an improved set of DNA barcode sequences, which may be referred to herein as “Pro-MID” barcodes (Pro-MID refers to “proprietary multiplex identification”). In an embodiment of the disclosure, Pro-MID barcodes comprise a plurality of barcode sequences, each sequence of the plurality of sequences characterized by any combination of at least four of the following criteria: (a) no repeated nucleotides of more than 2 nucleotides in length (by way of example, a sequence comprising AA is allowed but AAA is not); (b) GC content of between 30% and 60%; (c) at least 4 non-aligned nucleotides between each pair of Pro-MID barcodes; (d) no common sub-sequence of more than 8 nucleotides in length between any pair of Pro-MID barcodes; (e) a length of a palindrome if present is, optionally, at most 9 nucleotides in length, at most 8 nucleotides in length, at most 7 nucleotides in length, or at most 6 nucleotides in length; and (f) no reverse complementary sequences of more than 8 nucleotides in length between any pair of Pro-MID barcodes. Pro-MID barcodes are optionally between 7 and 15 nucleotides in length, between 7 and 9 nucleotides in length, 8 nucleotides in length, between 10 and 12 nucleotides in length, or 11 nucleotides in length. Optionally, each sequence of a set of Pro-MID barcodes is characterized by all of criteria (a) through (f) noted above. Optionally, a set of pro-MID barcodes consists of ninety-six (96) unique barcodes. In an embodiment of the disclosure, a barcode comprised in a HiCRISPR primer is a Pro-MID barcode according to an embodiment of the disclosure.

An aspect of the disclosure relates to providing a kit comprising PCR plates in which reaction wells comprised in the PCR plates are pre-loaded with HiCRISPR primers in accordance with embodiment of the disclosure, and further pre-loaded with a polymerase, free nucleotides, and buffering agents appropriate for performing a PCR.

An aspect of the disclosure relates to providing a two-step PCR method comprising: a first PCR reaction for spacer amplification using primers comprising a DR targeting region and a universal tail, such that product of the first PCR reaction comprises the universal tail; and a second PCR reaction for amplifying the spacer region comprised in the PCR products of the first PCR reaction, using primers comprising a targeting region complementary to the universal tail. Optionally, the primers used in the second PCR reaction further comprise one or more of a barcode, and an adapter having a sequence that makes the PCR product of the second PCR reaction compatible with an NGS. For convenience of presentation, the two-step PCR method in accordance with an embodiment of the disclosure may be referred to herein as “dual additive PCR”.

In an embodiment of the disclosure, the two steps of the dual additive PCR method are performed in a single reaction volume containing both a first primer pair for the first PCR reaction and a second primer pair for the second PCR reaction. Optionally, the annealing temperature for the first primer pair is higher than the annealing temperature of the second primer pair, such that the second primer pair do not anneal to their target regions in the template DNA during the first PCR reaction. For convenience of presentation, a dual additive PCR performed in a single reaction volume in accordance with an embodiment of the disclosure may be referred to herein as a “single tube dual PCR” or “std-PCR”.

An aspect of the disclosure relates to providing a method for phylogenetic analysis of bacterial samples based on a profile of spacers present in the CRISPR array of the bacterial samples.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins. The term “well-specific” as used herein is to be understood to mean specific to an individual reaction well for performing a PCR reaction, by way of example wells in a PCR plate.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1A shows a schematic representation of a CRISPR array;

FIG. 1B shows a detailed schematic representation of a region of a CRISPR array of M. tuberculosis;

FIG. 2 shows a schematic representation of HiCRISPR primers and their application in PCR in accordance with an embodiment of the disclosure;

FIG. 3 shows a flow diagram of a HiCRISPR method in accordance with an embodiment of the disclosure;

FIGS. 4A and 4B show exemplary products of a PCR using a HiCRISPR primer in accordance with an embodiment of the disclosure; and

FIG. 5 shows spacer profiles of M. tuberculosis samples prepared by a HiCRISPR method in accordance with an embodiment of the disclosure;

FIG. 6 shows a flow diagram of a dual additive PCR method in accordance with an embodiment of the disclosure;

FIGS. 7A and 7B shows a schematic representation of a dual additive PCR method in accordance with an embodiment of the disclosure;

FIG. 8 shows a schematic representation of an example PCR product of a dual additive PCR method in accordance with an embodiment of the disclosure;

FIG. 9 shows results of a % abundance of reads characterized by a given barcode pair for a plurality of PCR products generated by a dual additive PCR method in accordance with an embodiment of the disclosure;

FIG. 10 shows a flow diagram of a SPACERome-based phylogeny method in accordance with an embodiment of the disclosure;

FIG. 11A shows an example phylogenetic tree generated by a Codon Tree method; and

FIG. 11B shows an example phylogenetic tree generated by a SPACERome-based phylogeny method in accordance with an embodiment of the disclosure.

### DETAILED DESCRIPTION

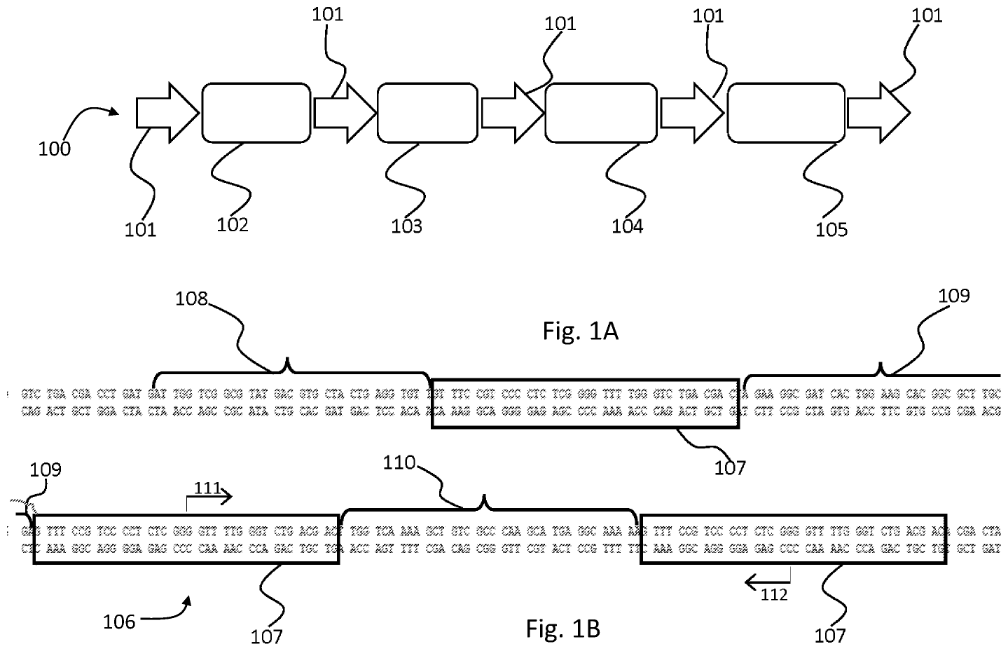

FIG. 1A shows a schematic representation of a CRISPR array within, for example, a bacterium 100. The Clustered Regularly Interspaced Short Palindromic Repeats or CRISPR is formed as a result of the CRISPR/Cas (CRISPR associated protein) system, a prokaryotic immune system that confers adaptive resistance as a form of acquired immunity to foreign genetic elements such as plasmids and phages. CRISPR Direct Repeats (DR) 101 are segments of prokaryotic genomic DNA containing short, repetitive base sequences. Each DR 101 is followed by a spacer 102 comprising a DNA sequence that is presumed to have originated from previous exposures to foreign DNA that was integrated into the CRISPR array by the CRISPR/Cas system. In addition, small clusters of cas (CRISPR-associated) genes (not shown) are located next to the CRISPR array. Four separate spacers 102-105 are shown in FIG. 1A, each spacer having a unique DNA sequence.

Some microbial species, such as M. tuberculosis, have a single CRISPR array. Other microbial species have a plurality of CRISPR arrays. Within a given CRISPR array, its DR consensus sequences are typically conserved. However, different CRISPR arrays within a same microbe or microbial species typically have different DR consensus sequences. A typical CRISPR array has less than 50 spacers. A DR typically range in size from 28 base pairs to 37 base pairs, though there can be as few as 23 base pairs and as many as 55 base pairs. The size of spacers in a CRISPR array is typically 32 base pairs to 38 base pairs, though there can be as few as 23 base pairs and as many as 55 base pairs. New spacers can appear as part of an immune response to, by way of example, phage infection. However, some microbial species, including M. tuberculosis, have a partially defective CRISPR/Cas system that is incapable of executing an adaptation step to add more spacers. A given CRISPR array from such species typically comprises spacers selected from a finite set of possible spacers. By way of example, CRISPR arrays of all known strains of M. tuberculosis have at most 43 spacers, or a subset selected from those 43 spacers.

FIG. 1B schematically shows a portion of a CRISPR array 106 of M. tuberculosis. Three DRs 107 are shown in FIG. 1B, and as can be seen in the figure within black squares, the sequence of these DRs are conserved as

(SEQ ID NO: 1)
GTTTCCGTCCCCTCTCGGGGTTTTGGGTCTGACGAC

and its complementary segment

(SEQ ID NO: 2)
GTCGTCAGACCCAAAACCCCGAGAGGGGACGGAAAC.

The portion of CRISPR array 106 as schematically shown in FIG. 1B comprises three spacer regions 108, 109, and 110. Also schematically shown are approximate binding sites for a pair of PCR primers, a forward primer 111 and a reverse primer 112, configured to amplify spacers found in CRISPR array 106. Each of primers 111, 112 comprises a targeting region that matches a portion of DR 107. As shown in FIG. 1B, forward primer 111 comprises a portion of SEQ ID NO:1 as a targeting region and reverse primer 112 comprises a portion of SEQ ID NO:2 as a targeting region. An exemplary pair of primers for amplifying spacer-comprising fragments from M. tuberculosis CRISPR array 106 are known in the art as “DRa” (alternatively “DR1”) and “DRb” (alternatively “DR2”) primers. The DR1 primer consists of a DR targeting region having the sequence GGTTTTGGGTCTGACGAC (SEQ ID NO: 3), which is the last 18 nucleotides at the 3′ end of the DR (SEQ ID NO: 1) in the M. tuberculosis CRISPR array. The DR2 primer consists of a DR targeting region having the sequence having the sequence CCGAGAGGGGACGGAAAC (SEQ ID NO: 4), which is the last 18 nucleotides at the 3′ end of the complementary DR sequence (SEQ ID NO: 2) found in the M. tuberculosis CRISPR array.

In accordance with an embodiment of the disclosure, a HiCRISPR primer comprises: (1) a barcode; and (2) a DR targeting region consisting of a sequence complementary to a portion of a DR comprised in a CRISPR array.

Sequences of DRs are usually species-specific. Therefore, in accordance with an embodiment of the disclosure, a DR targeting region comprised in a HiCRISPR primer is designed or selected based on the specific microbe species being analyzed. The sequence of DRs in the CRISPR array of many clinically or industrially relevant microbes have been characterized, including for Yersinia species, Erwinia amylovora, Escherichia coli, Salmonella enterica, M. tuberculosis, Campylobacter, Acinetobacter baumannii, group A Streptococcus, Lactobacillus gasseri and Bifidobacterium, and software tools such as “CRISPRfinder program online” or “CRISPRdb” from the University of South Paris are available for use in designing a sequence for a DR targeting region comprised in a HiCRISPR primer that is appropriate for a given microbe species selected to be characterized. As such, it would be reasonably expected that a HiCRISPR primer in accordance with an embodiment of the disclosure can be prepared and that a HiCRISPR method in accordance with an embodiment of the disclosure can be performed for any CRISPR array of any microbe provided that the DR sequence of the CRISPR array is known.

In an embodiment of the disclosure, a barcode comprised in a HiCRISPR primer consists of one barcode selected from a set of unique barcodes for indexing and/or multiplexing DNA fragments. In an embodiment of the disclosure, each barcode of the set of barcodes are designed to not only be unique but also sufficiently distinct so that the likelihood of barcode misidentification caused by unintended barcode modification resulting from insertions, deletions and substitutions during PCR amplification, as well base-calling errors during NGS, is sufficiently low. Optionally, the barcode is between 8 and 15 nucleotides in length, optionally 11 nucleotides in length. Optionally, the barcode is one out of a set consisting of 96 barcodes, by way of example NEBNext® Multiplex Oligos, or Pro-MID barcodes in accordance with an embodiment of the disclosure.

Optionally, the barcode and the targeting region are directly adjacent to each other, with no intervening nucleotides between them. Optionally, the barcode and the targeting region are separated by intervening nucleotides. Optionally, the intervening nucleotides comprise no more than four nucleotides.

In an embodiment of the disclosure, a HiCRISPR primer consists of a barcode and a DR targeting region arranged in a 5′ to 3′ direction, as well as, optionally, ten or less additional nucleotides, five or less additional nucleotides, one additional nucleotide, or no additional nucleotides. Optionally, a HiCRISPR primer consists of between zero and four capping nucleotides, a barcode, an intervening region consisting of between zero and four nucleotides, and a DR targeting region arranged in a 5′ to 3′ direction. Optionally, the capping nucleotides consists of a single guanine. Optionally, a HiCRISPR primer consists of a guanine nucleotide, a barcode, and a DR targeting region arranged in a 5′ to 3′ direction.

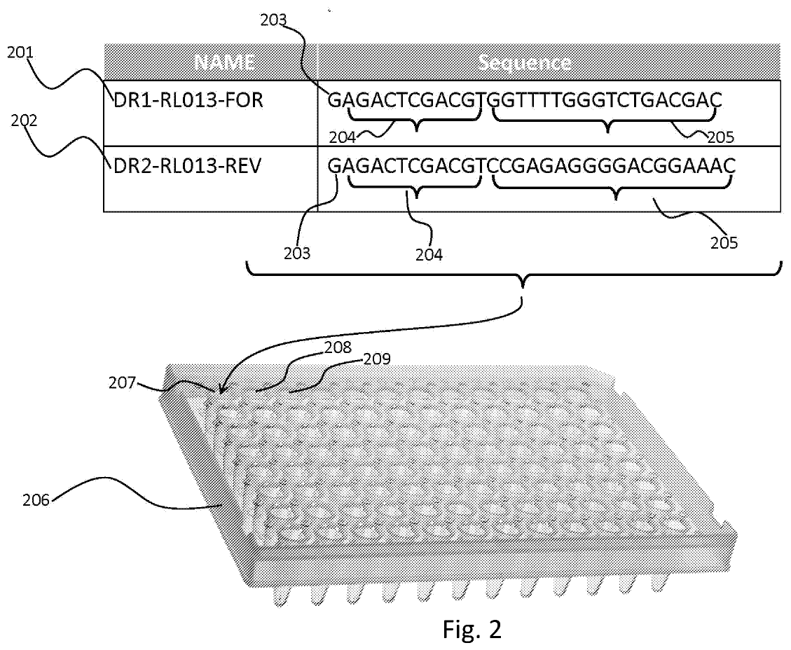

FIG. 2 shows an exemplary pair of HiCRISPR primers, a “DR1” HiCRISPR forward primer 201 and a “DR2” HiCRISPR reverse primer 202. DR1 HiCRISPR primer 201 (having a name DR1-RL013-FOR as shown in FIG. 2) consists of the sequence GAGACTCGACGT GGTTTTGGGTCTGACGAC (SEQ ID NO:5), which encodes the following three regions arranged in a 5′ to 3′ direction: a single guanine nucleotide at the 5′ end; followed by a barcode 204 of eleven nucleotides in length having the sequence AGACTCGACGT (SEQ ID NO: 6), and further followed by a DR targeting region 205 having the DR1 sequence (SEQ ID:3). The particular sequence of barcode 204 as shown in FIG. 2 is a selection from a set of barcodes as described in U.S. patent publication 2011/003701 A1.

DR2 HiCRISPR primer 202 (having a name DR2-RL013-REV as shown in FIG. 2) comprises the sequence GAGACTCGACGTCCGAGAGGGGACGGAAAC (SEQ ID NO:7), which encodes the following three regions arranged in a 5′ to 3′ direction: a single guanine; followed by a barcode 204 of eleven nucleotides in length having the sequence AGACTCGACGT (SEQ ID NO: 8); and further followed by a DR targeting region 205 having the DR2 sequence (SEQ ID NO: 4).

An NGS system typically requires that DNA strands be flanked by an adapter characterized by a known sequence, which makes the strands compatible with, and able to be sequenced by, the NGS system. In some NGS systems, by way of example NGS systems by Illumina®, a DNA sample is deposited into a flow cell that includes a “lawn” of DNA oligonucleotides bound to its bottom surface. The surface-bound oligonucleotides include a region that is complementary to at least a portion of the sequence of a compatible adapter, such that DNA strands having the compatible adapter are captured by the lawn and retained for sequencing. In other NGS systems, by way of example NGS systems by Ion-Torrent®, the adaptor sequence allow binding to the DNA samples to the surface of beads lined with matching oligonucleotides.

In an embodiment of the disclosure, a HiCRISPR primer comprises an adapter compatible with a NGS system in addition to a barcode and a DR targeting region. By way of example, the NGS system for which the adapter is compatible is a commercial NGS system from, by way of example Illumina® (MiSeq® and others), Roche® (454®), and Ion-Torrent® for which compatible adapters are established and known. For convenience of presentation, a HiCRISPR primer comprising an adapter may be referred to herein as a “HiCRISPR(adp+)” primer, and a HiCRISPR primer that does not comprise an adapter may be referred to herein as a “HiCRISPR(apd−)” primer.

Optionally, a HiCRISPR(adp+) primer consists of a DNA sequence encoding an adapter, a barcode, and a DR targeting region arranged in a 5′ to 3′ direction, as well as, optionally, ten or less additional nucleotides, five or less additional nucleotides, one additional nucleotide, or no additional nucleotides. Optionally, a HiCRISPR(adp+) primer consists of an adapter, a barcode, an intervening region consisting of between one and four nucleotides, and a DR targeting region arranged in a 5′ to 3′ direction.

An aspect of the disclosure relates to providing an improved set of barcodes, referred to herein as Pro-MID barcodes. In an embodiment of the disclosure, a set of Pro-MID barcodes comprises a plurality of barcodes, each barcode of the plurality of barcodes characterized by at least four of the following criteria:
a) No repeated nucleotides of more than 2 nucleotides in length;
b) GC content between 30% and 60%;
c) Each pair of Pro-MID barcodes comprises at least 4 nucleotide positions where the respective bases are different;
d) No pair of Pro-MID barcodes share a common sub-sequence of more than 8 nucleotides in length;
e) A maximum length of a palindrome within a Pro-MID barcode is, optionally, 9 nucleotides in length, 8 nucleotides in length, 7 nucleotides in length, or 6 nucleotides in length; and
f) Maximum length of a reverse complementary sub-sequence between any pair of Pro-MID barcodes is 8 nucleotides.

Optionally, a set of Pro-MID barcodes comprises a plurality of barcodes, each barcode of the plurality of barcodes characterized by all of criteria (a) through (f). Pro-MID barcodes are optionally between 7 and 15 nucleotides in length, between 7 and 9 nucleotides in length, 8 nucleotides in length, between 10 and 12 nucleotides in length, or 11 nucleotides in length. In a particular embodiment, a set of Pro-MID barcodes comprises a plurality of barcodes that are each 11 barcodes in length, each barcode of the plurality of barcodes characterized by all of criteria (a) through (f). Pro-MID barcodes in accordance with an embodiment of the disclosure represent an improvement over other barcode sets known in the art due to one or more of the following advantages: less prone to generate secondary structures; less prone to hybridizing with another Pro-MID barcode, having a GC content that is better suited for NGS systems, less prone to barcode misidentification, and being more suitable for low complexity amplicon sequencing. These advantages are expected to provide more accurate base-calling and sequence data when applied to NGS.

There is provided in accordance with an embodiment of the disclosure an exemplary set of 96 Pro-MID barcodes (SEQ ID NOS:9-104) in which each barcode is eleven (11) nucleotides in length and is characterized by all of criteria (a) through (f) listed above. There is provided in accordance with an embodiment of the disclosure a second exemplary set of 96 Pro-MID barcodes (SEQ ID NOS: 105-200) in which each barcode is eight (8) nucleotides in length and is characterized by all of criteria (a) through (f) listed above. Table 1 below shows comparisons of various sequence parameters relating to barcode sequence, between a set of barcodes (“Roche/454 barcodes”) known in the art and as described in U.S. patent publication 2011/003701 A1, and the set of Pro-MID barcodes (both the 11-nucleotide version and the 8-nucleotide version). The set of Roche/454 barcodes comprise a total of 133 barcodes, each barcode being eleven nucleotides in length. Sequences of individual barcodes of the Roche/454 barcodes can be found as SEQ ID NOS 1-133 in U.S. patent publication 2011/003701 A1. 96 out of the 133 Roche/454 barcodes, which were selected for use in Example 1 described hereinbelow, were included for the comparisons shown in Table 1.

TABLE 1
Comparison with Roche/454 barcodes

| | Roche/454 (96 barcodes, | |
|---|---|---|
| | 4560 possible barcode | Pro-MID (96 barcodes; |
| Parameter | pairs) | 4560 possible barcode pairs) |
| GC content | between 18% and 73% | Between 36% and 54% |
| | (median: 45%) | (median: 54%) |
| Barcodes with a palindrome of 6 | 17 out of 96 (17.7%) | 7 out of 96 (7.2%) |
| nucleotides in length | | |
| Barcodes with a palindrome of 8 | 3 out of 96 (3.2%) | 0 out of 96 (0%) |
| nucleotides in length | | |
| Barcodes with a palindrome of 10 | 1 out of 96 (1.1%) | 0 out of 96 (0%) |
| nucleotides in length | | |
| Barcodes with a palindrome of 6 | 21 out of 96 (21.9%) | 7 out of 96 (7.3%) |
| nucleotides or more in length | | |
| Maximum palindrome length | 10 | 6 |
| Barcode pairs sharing a sub- | 217 out of 4560 (4.7%) | 35 out of 4560 (0.8%) |
| sequence of 5 nucleotides in length | | |
| Barcode pairs sharing a sub- | 59 out of 4560 (0.7%) | 10 out of 4560 (0.2%) |
| sequence of 6 nucleotides in length | | |
| Barcode pairs sharing a sub- | 12 out of 4560 (0.14%) | 6 out of 4560 (0.13%) |
| sequence of 7 nucleotides in length | | |
| Barcode pairs sharing a sub- | 7 out of 4560 (0.08%) | 1 out of 4568 (0.02%) |
| sequence of 8 nucleotides in length | | |
| Barcode pairs sharing a sub- | 290 out of 4560 (6.4%) | 52 out of 4560 (1.1%) |
| sequence of 5 nucleotides or more | | |
| in length | | |
| Maximum length of shared sub- | 8 | 8 |
| sequences between barcode pairs | | |
| Number of nucleotide position | Between 4 and 10 | Between 4 and 11 nucleotide |
| where the respective bases are | nucleotide positions | positions (median: 8 |
| different between barcode pairs | (median: 7 nucleotide | nucleotide positions) |
| | positions) | |

Alternatively or additionally to criteria a) through f) noted above, a set of Pro-MID barcodes in accordance with an embodiment of the disclosure are optionally characterized by one or more of the following criteria: a GC content of between 35% and 55%; less than 10% or less than 15% of Pro-MID barcodes in a set of Pro-MID barcodes comprise a palindrome of 6 or more nucleotides in length; the maximum length of a palindrome comprised in a Pro-MID barcode is 6 nucleotides; less than 5%, less than 3% or less than 2% of pairs of Pro-MID barcodes out of all possible combinations of Pro-MID barcode pairs share a same sub-sequence of 5 nucleotides or more in length.

A Pro-MID barcode in accordance with an embodiment of the disclosure is compatible for use in a HiCRISPR method, and for being comprised as a barcode in a HiCRISPR primer. As such, there is provided in accordance with an aspect of the disclosure a HiCRISPR primer wherein the barcode is a Pro-MID barcode. It will be appreciated that the use of Pro-MID barcodes is not limited to use in a HiCRISPR method or being comprised in a HiCRISPR primer. Pro-MID barcodes may be used in other applications where DNA barcodes are useful. It will also be appreciated that a primer comprising a Pro-MID barcode and an appropriately selected targeting region can be applied to a wide variety of applications beyond CRISPR-typing, where high-throughput genetic analysis of biological samples, optionally using NGS, is useful. Examples of application for primers comprising Pro-MID barcodes in accordance with an embodiment of the disclosure include: validating identity of natural products (including wild-harvested fish and plants); tracking genetic identity of agricultural products; epidemiologically tracking clinically relevant microbial species and/or strains (for example in a hospital, a city, or a country); identifying genomic factors contributing to tumorigenesis; and gut microbiome characterization of subjects.

Reference is made back to FIG. 2. In accordance with an embodiment of the disclosure, in a HiCRISPR method, HiCRISPR primers are used in PCR reactions to amplify portions of a CRISPR array comprising spacers from a plurality of microbial DNA isolates, optionally in parallel, by way of example by loading each reaction well in a 12-well strip or a 96-well plate with a different microbial DNA isolate.

FIG. 2 schematically shows a 96-well plate 206 comprising reaction wells 207, 208, and 209 as well as ninety-three other reaction wells, with each reaction well being loaded with primers comprising a well-specific barcode or barcode pair. By way of example, reaction well 207 is loaded with forward HiCRISPR primer 201 and reverse HiCRISPR primer 202, which both comprise a same barcode AGACTCGACGT (SEQ ID NO: 8). Reaction well 208 is loaded with a HiCRISPR primer pair comprising a barcode identical to each other but different from the barcode comprised in the HiCRIPSR primers 201, 202 loaded in reaction 207, and the barcode comprised in the HiCRISPR primers loaded in reaction well 209 is different from either of the barcodes found in reactions wells 207 and 208. In an embodiment of the disclosure, each reaction well in 96-well PCR plate 206 is also loaded with a PCR reagent mix comprising a polymerase enzyme, free nucleotides and buffering agents appropriate for a PCR. Optionally, the PCR reagent mix and the HiCRISPR primers are pre-loaded and lyophilized, and reconstituted when appropriate with water and further loaded with a microbial DNA isolate prior to a PCR being initiated. By way of example, hot start proof reading enzymes such as KOD Hot Start® (Novagen®) and FastStart® High Fidelity System (Roche®) may be used for the PCR reaction. Whereas FIG. 2 shows a 96-well plate, other formats such as strips or plates suitable for 12, 48 or 384 samples may be used.

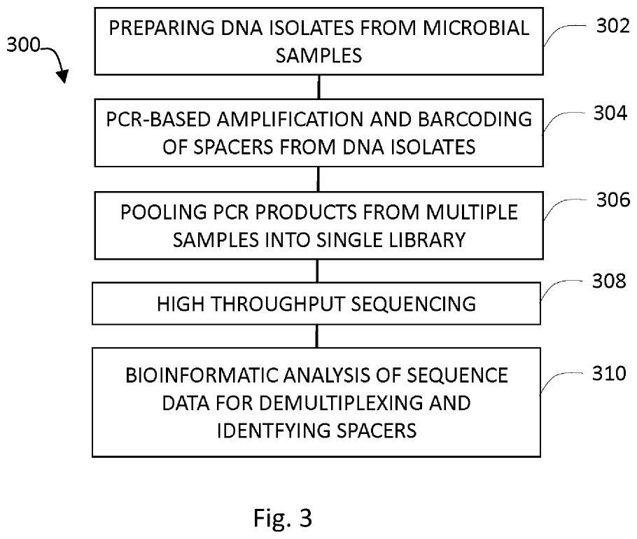

FIG. 3 shows a flowchart 300 illustrating steps of a HiCRISPR method in accordance with an embodiment of the disclosure. As shown in FIG. 3, HiCRISPR method 300 in accordance with an embodiment of the disclosure comprises: a block 302 comprising preparation of microbial DNA isolates, a block 304 comprising PCR-based amplification and barcoding of spacer from the microbial DNA isolates, a block 306 comprising pooling PCR products prepared from each of the microbial DNA isolates, a block 308 comprising NGS of the pooled PCR products, and a block 310 comprising bioinformatic analysis of sequence data from the NGS to demultiplex sequences data generated from each microbial DNA isolate and identify sequences that encode spacers.

In an embodiment of the disclosure, block 302 comprises preparation of crude or purified DNA isolates from a plurality of microbial samples. In an embodiment of the disclosure, each microbial sample is a genetically pure microbial sample, by way of example a sample of a microbial colony grown from a single cell. Optionally, microbial DNA isolates are obtained by incubation of microbial samples in water at 95° C. for 10 min followed by centrifugation. Optionally, microbial DNA isolates are isolated, purified and prepared for PCR usage by utilizing one of commonly available commercial kits. Particular protocols and kits optimized for known microbial species are known in the art. By way of example, preparation of DNA isolates from M. tuberculosis is achieved by the DNA extraction protocol described in the Geno Type Mycobacterium CM Ver. 1.0, produced by Hain Lifescience GmbH, Germany.

In an embodiment of the disclosure, Block 304 comprises PCR-based amplification and barcoding of spacers found in CRISPR arrays comprised in each of a plurality of microbial DNA isolates.

In an embodiment of the disclosure, the PCR step comprises, for each of a plurality of reaction wells, adding a polymerase enzyme, free nucleotides, buffers, and a pair of well-specific HiCRISPR primers, together with one of the microbial DNA isolates produced in block 302. In an alternative embodiment, a HiCRISPR kit is provided in which each reaction well of a PCR plate is pre-loaded with a pair of well-specific HiCRISPR primers. Each well is optionally further preloaded with a PCR reaction mix comprising a polymerase enzyme, nucleotides and buffers. By using a HiCRISPR kit with preloaded HiCRISPR primers and a PCR reaction mix as described above, PCR step 304 may be simplified to require only the addition of DNA isolates in the wells and water as needed to reconstitute the preloaded constituents to an appropriate concentration, prior to placing the loaded kit into a PCR thermocycler.

In an embodiment of the disclosure, well-specific HiCRISPR primers loaded in each reaction well are characterized by a unique barcode or a unique pair of barcodes. In a situation where each reaction well is loaded with a microbial DNA isolate prepared from a different microbial sample, well-specific primers or barcodes is also understood to be sample-specific. Optionally, each primer of a pair of well-specific HiCRISPR primers comprises a same barcode. As a result, all PCR products generated by the HiCRISPR primer pair would comprises two regions comprising the barcode sequence. In a situation where the barcode is one out of a set of 96 unique barcodes, having each pair of primers have the same barcode allows a maximum of 96 PCR reactions to be multiplexed. Alternatively, a pair of well-specific HiCRISPR primers may be characterized by a unique pair of barcodes. If a forward primer comprises a first barcode selected from a set of barcodes and a reverse primer comprises a second barcode selected from the set of barcodes, then the resulting PCR products would comprise both the first and second barcodes. For situations in which a set of barcodes consists of 96 unique barcodes, characterizing PCR products with a pair of barcodes by having the forward and reverse primers each comprise a different barcode allows a maximum of 4560 (equal to 96×95/2) PCR reactions to be multiplexed.

In an embodiment of the disclosure, the PCR step includes adding a positive control, for example, in a particular well including a DNA isolate of a specific strain of the bacterial species being analyzed in which the sequence of the CRISPR array has already been elucidated. In an embodiment of the disclosure, a HiCRISPR PCR kit may be provided pre-loaded with a species-specific positive control. Additionally, a negative control may also be included in the PCR amplification whereby one well is left devoid of bacterial DNA.

Any suitable thermocycler may be used for HiCRISPR. In an embodiment of the disclosure, Real-Time PCR may be used in order to monitor the progress of the amplifications and perform melting curve analyses to provide an indication as to the success or failure of each reaction.

In an embodiment of the disclosure, HiCRISPR method 300 comprises a block 306 comprising pooling of amplified, barcoded spacers produced by the PCR reaction from each of the plurality of microbial DNA isolates into a single DNA library, which may be referred to herein as a “multiplexed spacer library”. In an embodiment of the disclosure, following PCR in a standard 96-well PCR plate, a sample (by way of example 2-4 microliters) of the reaction product is removed from each well and combined together to generate a mixture that represents all the microbial DNA isolates. In an embodiment, the multiplexed spacer library may undergo column purification before being sent for sequencing and sequence analysis.

In a block 308 in accordance with an embodiment of the disclosure, a multiplexed spacer library is sequenced by NGS. Examples of NGS systems in the art include MiSeq® by Illumina®, 454® sequencing by Roche®, Ion-torrent® sequencing, and SOLiD (Sequencing by Oligonucleotide Ligation and Detection) by Life Technologies®. Optionally, for embodiments in which a HiCRISPR(adp-) primer is used in the PCR step (block 304), an adapter is ligated to the amplified, barcoded spacers comprised in the multiplexed spacer library before being processed by a NGS system for which the adapter is compatible. Alternatively, for embodiments in which the PCR step (block 304) is performed with HiCRISPR(adp+) primers, a separate ligation step to add the adapter is not needed because the amplified spacers are linked with an adapter during the PCR amplification process.

In a block 310 in accordance with an embodiment of the disclosure, sequencing data produced by NGS is analyzed using bioinformatic processes to create a profile of the specific spacers comprised in the CRISPR array of each of the plurality of analyzed microbial DNA isolates. In an embodiment of the disclosure, sequencing data, by way of example fastq files, generated by an NGS system during the NGS step (block 308) are demultiplex based on barcodes so that sequences of PCR products produced in a same reaction well during PCR are sorted together into their respective groups. Each sorted set of sequence data, which represents a particular reaction well and thus a particular microbial DNA sample, is then analyzed to identify sequences encoding spacers and produce a profile of all unique spacers associated with the particular microbial DNA sample. This profile is designated as a “spacer profile” associated with, and serving as identification for, the particular microbial sample from which the microbial DNA isolate was produced.

Spacers may be identified from NGS sequence data using a computer-based sequence analysis program in a number of ways. Optionally, NGS sequence data generated from a sample of a given microbial species is compared against sequences of known spacers for the given species, and portions within the NGS sequence data that are homologous to a known spacer are designated as a spacer. Alternatively or additionally, spacers are identified based on the sequence of at least a portion of the DR of the CRISPR array from which the NGS sequence data was generated. Provided that the NGS sequence data was generated from a PCR using a pair of HiCRISPR primers in accordance with an embodiment of the disclosure, spacers comprised in the NGS sequence would be expected to be flanked by at least the sequence of the DR targeting regions comprised in the HiCRISPR primers. By way of example, portions of the NGS sequence data characterized by a predetermined range of nucleotide length and flanked by at least a portion of the DR may be designated as a spacer. This second method of identifying spacers based on the DR allows for identification of new spacers, without being limited to searching for previously known spacers.

Optionally, the spacer profile is in the form of an N-dimensional binary vector, which may be represented as a string of N ones and zeros, in which each digit represents the presence (1) or absence (0) of a given unique spacer out of a set of N unique spacers. A set of N unique spacers may be based on spacers identified from one or more multiplexed spacer libraries made in block 306, or may be based on unique spacers that have been previously characterized in a given microbial species. By way of example, there are 43 unique spacers that are known to be found in CRISPR arrays of M. tuberculosis, so that a CRISPR array of a given genetically pure sample of M. tuberculosis is expected to consist of some combination of these known 43 unique spacers. As such, in an embodiment of the disclosure, a spacer profile associated with a M. tuberculosis comprises a 43-dimensional binary vector, which may be expressed as a string of 43 ones and zeros, or translated into octal code.

In an embodiment of the disclosure, a PCR reaction performed in block 304 is performed with at least one additional pair of primers, which may be referred to as “supplemental classification primers” comprising a well-specific barcode and a targeting region encoded to amplify at least a portion of: a virulence gene, a lineage marker, and/or an antibiotic resistance gene. Typically, sequences of virulence and antibiotic resistance genes are species-specific. As such, a targeting region of supplemental classification primers are selected based on the species of the microbial sample. For embodiments in which the PCR reaction is performed with supplemental classification primers, the bioinformatic analysis performed in block 308 further comprises analyzing the demultiplexed sequence data to detect virulence and/or antibiotic resistance genes that may have been amplified from the respective microbial DNA samples by the supplemental classification primers. Optionally, correlation between presence of virulence and/or antibiotic resistance genes as detected by supplemental classification primers in a given microbial DNA sample and the corresponding spacer profile is analyzed to identify spacer profiles that predict or indicate a statistical likelihood of virulence or antibiotic resistance.

Microbes can exhibit antibiotic resistance by: expressing an enzyme that inactivates the antibiotic; mutation that renders an antibiotic binding site less suited for biding by the antibiotic; alteration of metabolic pathway; or reducing drug accumulation through decreasing drug permeability or increasing efflux of the drug. Antibiotic resistance can arise through mutation or horizontal gene transfer. Antibiotic resistance genes and mutations thereof for many clinically relevant microbes have been characterized and are known in the art. By way of example, sequences of previously characterized antibiotic resistance genes, or their mutations, may be searched for and selected using online resources such as the Antibiotic Resistance Gene Database.

Different strains within a microbial species may have different levels of virulence, which is a microbe's ability to infect or damage a host. Different strains may have differences in certain genetic elements, such as structural genes (“virulence genes”) or mobile genetic elements that produce differences in the strains' virulence. These genetic elements, which may be referred to as virulence factors, include genes that encode proteins that, for example, improve the microbe's ability to adhere to a host tissue or cell, enter a host cell through endocytosis, inhibit host immune response, or serve as or synthesize toxins. Examples of virulence genes include genes that encode adhesins, immunoglobulin proteases, GTPase modulators. Virulence genes for many clinically relevant microbes have been characterized and are known in the art.

Reaction wells of HiCRISPR PCR-kits in accordance with an embodiment of the disclosure as disclosed herein are optionally preloaded with supplemental classification primers for detecting a virulence gene or an antibiotic resistance gene, in addition to species-matched HiCRISPR primers and standard PCR reagents.

Reference is made back to FIG. 3. HiCRISPR method 300 in accordance with an embodiment of the disclosure may be applied to characterizing spacers from a plurality of different CRISPR arrays in each of a mixed microbial DNA isolate comprising DNA isolated from a biological sample comprising a plurality of different microbial species and/or strains.

With respect to block 302, a microbial DNA isolate is optionally prepared from a biological sample comprising a plurality of different microbial species and/or strains. By way of example, the biological sample may be a tissue sample, a fecal sample, a urine sample, a sputum sample, a soil sample, or a plant sample.

With respect to block 304 relating to PCR-based amplification and barcoding of spacers found in CRISPR arrays, each PCR reaction is optionally a “multiplexed PCR reaction”, in which a reaction well is loaded with a plurality of pairs of HiCRISPR primers to amplify spacers from a plurality of different CRISPR arrays, optionally CRISRP arrays found in different microbial species. Optionally, the multiplexed PCR reaction comprises between 2 and 100 distinct pairs of HiCRISPR primers to amplify primers from between 2 and 100 different CRISPR arrays. By way of example, each reaction well may be loaded with three different pairs of HiCRISPR primers: a first primer pair comprising DR target regions selected to amplify spacers comprised in a CRISPR array found in Escherichia coli, a second primer pair comprising DR target regions selected to amplify spacers comprised in a CRISPR array found in Salmonella enterica; and a third primer pair comprising DR target regions selected to amplify spacers comprised in a CRISPR array found in a Campylobacter species. As noted above, different CRISPR arrays are typically characterized by a different DR consensus sequence. In an embodiment of the disclosure, each HiCRISPR primer used in a multiplex PCR in a given reaction well comprises a unique DR targeting region. Moreover, the HiCRISPR primer loaded in each reaction well comprises well-specific barcodes in accordance with an embodiment of the specification.

After the multiplexed PCR is performed for each of the plurality of mixed microbial DNA isolates, sample from each PCR is pooled to form a multiplexed spacer library (block 306), and sequenced by NGS (block 308).

With respect to block 310 relating to bioinformatic analysis of the NGS sequence data, in a situation in which each microbial DNA isolate is prepared from a biological sample comprising a mixture of different microbial species and/or strains, and processed with multiplex PCR with a plurality of HiCRISPR primer pairs to amplify spacers from a plurality of different CRISPR arrays, the sequence of each PCR product would be expected to comprise the following in addition to one or more spacers: (1) a sequence of a barcode or a pair of barcodes identifying the reaction well in which the PCR produce was produced; and (2) sequences of DR targeting regions comprised in the forward and reverse HiCRISPR primers that produced the PCR product, which identify the CRISPR array from which the PCR product was amplified.

In accordance with an embodiment of the disclosure, NGS sequence data is demultiplexed based on barcodes to sort for sequence data of PCR products produced in a same reaction well, and thus amplified from the same biological sample. The sorted sequence data is then analyzed as described above to identify sequences encoding spacers. The set of spacer sequences identified thereby, in accordance with an embodiment of the disclosure, may vary greatly between different biological samples, or between similar biological samples collected at different times or from different sources, and thus may be useful as identification or a signature for the biological sample. For convenience of presentation, a set of spacers amplified, sequenced, and identified from a biological sample through multiplex PCR with a plurality of HiCRISPR primer pairs, in accordance with an embodiment of the disclosure, may be referred to as a “spacer profile” or “SPACERome” characterizing the biological sample or the source of the biological sample.

In an embodiment of the disclosure, the SPACERome comprises not only the presence or absence of particular spacers, but also a relative abundance of each spacer in a biological sample, based on the number of copies of each spacer that was sequenced from the multiplexed spacer library by NGS.

In an embodiment of the disclosure, the SPACERome comprises a relative abundance of different microbial species in a biological sample, based on the number of copies of distinctive DR sequences sequenced from the multiplexed spacer library.

Advantageously, a SPACERome can serve as an economical, high-throughput, and fast method to characterize the microbiome of a subject. Optionally, a SPACERome is generated from a plurality of biological samples from a same source such as a given subject over time, and can thus be used to track changes in a subject's microbiome over time, by way of example changes responsive to treatments such as administration of probiotic formulas or fecal microbiota transfer (FMT).

### EXAMPLE 1

A HiCRISPR method in accordance with an embodiment of the disclosure was carried out with samples from 96 microbial DNA isolates taken from M. tuberculosis samples. Ninety six (96) PCRs were performed to amplify spacers from the 96 M. tuberculosis DNA isolates. Each PCR reaction comprised one of the 96 M. tuberculosis DNA samples, a forward HiCRISPR primer comprising a DR1 sequence and a sample-specific barcode selected from a set of Roche/454 barcodes; a reverse HiCRISPR prime comprising a DR2 sequence and the same sample-specific barcode; and PCR reagents as appropriate. The PCR reactions were performed in a thermocycler programmed to consist of the following steps:
a) initiation step: 95° C. for 2 minutes;
b) denaturation step: 95° C. for 30 seconds;
c) annealing step: 55° C. for 30 seconds;
d) elongation step: 72° C. for 30 seconds;
e) repeat steps b-d for 40 cycles; and
f) final elongation step: 72° C. for 4 minutes.

The PCR was conducted as a quantitative PCR to monitor the progress of amplification, and any reaction in which the threshold cycle was greater than 25 was discarded from further analysis. A sample from each of the non-discarded reactions was pooled together to form a multiplexed spacer library, which was then processed with a ligation reaction to attach Illumina®—compatible adapters (about 60 base pairs in size) to each of 5′ and 3′ ends of the PCR products.

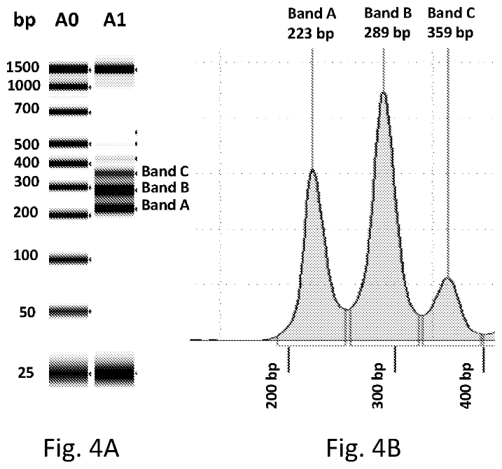

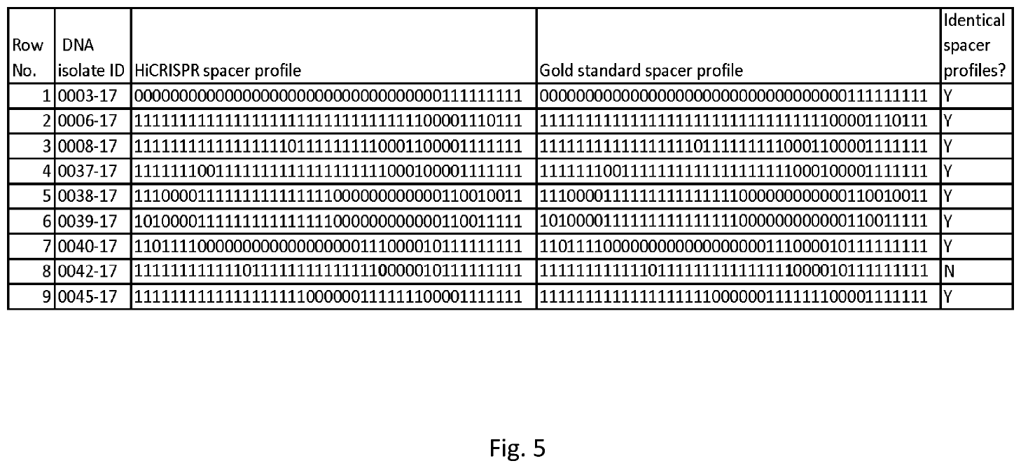

FIG. 4A shows a virtual gel electrophoresis view of two DNA samples (A0 and A1) analyzed by capillary electrophoresis (TapeStation System, Agilent). Sample A0 consists of a reference DNA ladder, and sample A1 consists of 25 bp and 1500 bp reference DNA molecules, as well as the multiplexed spacer library. The bands from sample A0 reflect reference DNA molecules of varying lengths, ranging in size from 25 bp to 1500 bp. Sample A1 comprised six bands out of which three were high-intensity bands (band A, band B, and band C) representing the major PCR products from the library and another three peaks representing minor PCR products from the library. The bands at 25 bp and 1500 bp reflect the added reference markers in Sample A1.

FIG. 4B shows a portion of a chromatogram (x-axis=base pairs; y-axis=DNA concentration) generated by the TapeStation System based on the capillary electrophoresis of Sample A1, which corresponds to the virtual gel electrophoresis view shown in FIG. 4A. FIG. 4B shows the portion of the chromatogram between about 180 base pairs (bp) and about 420 bp, which includes three peaks corresponding to bands A-C as shown in FIG. 4A. The three peaks are labeled as band A, band B, and band C accordingly. Based on the respective peak maximum corresponding to each of bands A-C, the PCR products at band A were estimated to have a maximal size of 223 bp, the PCR products at band B were estimated to have a maximal size of 289 bp, and the PCR products at band C were estimated to have a maximal size of 359 bp. Out of the total integrated area under the respective intensity curves for all six bands representing the PCR products comprised in the multiplexed spacer library comprised in Sample A1, band A represented about 32% of the total integrated area, band B represented about 51% of the total integrated area, and band C represented about 14% of the total area. Therefore, the PCR products represented by bands A-C account for about 97% (32%+51%+14%) of the total amount of DNA amplified in the PCR. Based on their respective sizes (taking into account the additional 120 bp for the two 60 bp adapters added by ligation) band A was determined to consist of amplified CRISPR array segments having one spacer, band B was determined to consist of amplified CRISPR array segments having two spacers, and band C was determined to consist of amplified CRISPR array segments having three spacers.

### EXAMPLE 2

A HiCRISPR method in accordance with an embodiment of the disclosure was carried out with samples from 145 microbial DNA isolates taken from M. tuberculosis samples, for CRISPR-typing each of the 145 microbial DNA isolates. The PCRs were performed as described with respect to Example 1. The HiCRISPR method for the 145 samples was carried out in three separate batches of 25 samples, 85 samples, and 35 samples, respectively. For each batch, samples from the PCRs were pooled to form a multiplexed spacer library. Each multiplexed spacer library was processed with a ligation reaction to attach MiSeq®-compatible adapters to the PCR products comprised therein, then sequenced with a MiSeq® NGS system. A single run of MiSeq® sequencing (nano-run 150X2; paired end) yielded between 700,000 to 1.2 million reads. The DNA sequence data generated by the MiSeq® system was analyzed to demultiplex the sequences from each microbial DNA isolate based on barcodes, identify sequences encoding spacers, and generate a spacer profile (a “HiCRISPR spacer profile”) for each of the microbial DNA isolates processed in the given batch.

Each of the same 145 microbial DNA isolates were separately processed using a “gold standard” hybridization-based spoligotyping method referred to above, in five batches of up to 35 samples, to generate another spacer profile (a “gold standard spacer profile”). The HiCRISPR spacer profiles and the gold standard spacer profiles each included, in total, 43 unique spacers. As such, each spacer profile (whether HiCRISPR or gold standard) was expressed as a binary feature vector of 43 dimensions, each dimension corresponding to one of the 43 unique spacers, and each dimension having either a value of “0” signifying absence of a given unique spacer in the spacer profile or a value of “1” signifying presence of the given spacer in the spacer profile.

For each M. tuberculosis sample, the sample's HiCRISPR spacer profile was compared with the corresponding gold standard spacer profile. A comparison of the respective spacer profiles for nine of the 145 samples are shown in FIG. 5. By way of example, as shown in FIG. 5, the HiCRISPR spacer profile and the gold standard spacer profile for sample ID 0042-17 (in row 7) were not identical: while the spacer represented by the 27th digit in the spacer profile was deemed to be absent (indicated by a bold “0”) according to the HiCRISPR spacer profile, the same spacer was deemed to be present (indicated by a bold “1”) according to the gold-standard spacer profile. For each the other eight samples shown in FIG. 5, both CRISPR-typing methods produced identical spacer profiles. Out of the total 145 microbial DNA isolates, the HiCRISPR spacer profile and the gold standard spacer profile were identical in 141 of the isolates, thus showing that the HiCRISPR method was able to CRISPR-type the microbes with 97% accuracy compared to the gold standard spoligotyping method. Moreover, comparing the two spacer profiles for the 145 samples amounts to comparing the presence or absence of 6235 spacers (145 samples×43 spacers in the spacer profile of each sample). Out of these 6235 spacer comparisons, it was found that 6231 spacers were correctly identified by the HiCRISPR method with respect to the gold standard method, representing a spacer identification accuracy of 99.93%. Further analysis of some of the above-noted discrepancies between the spacers identified by the HiCRISPR method and spacers identified by the spoligotyping method revealed that the source of most of the discrepancies studies were human errors made in the spoligotyping method (by way of example a misreading of results).

Moreover, while each batch for gold standard spoligotyping, as noted above, typically takes about 3 or 4 days to complete, each batch processed in accordance with an embodiment of the HiCRISPR method was completed in one or two days using relatively inexpensive, readily available equipment and reagents for PCR and NGS.

The above result indicates that, unlike other alternative CRISPR-typing methods (such as spacer oligonucleotide-conjugated microspheres, MALDI-TOF MS, whole-genome sequencing, and DNA microarrays) that have been developed subsequent to the gold-standard spoligotyping method, a HiCRISPR method in accordance with an embodiment of the disclosure is able to serve as a faster, higher throughput, cost-effective alternative to (and replacement for) the gold standard spoligotyping method.

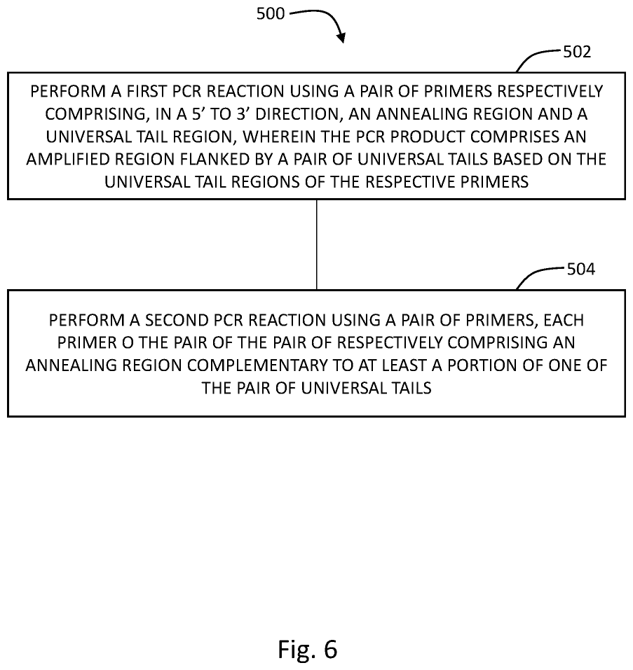

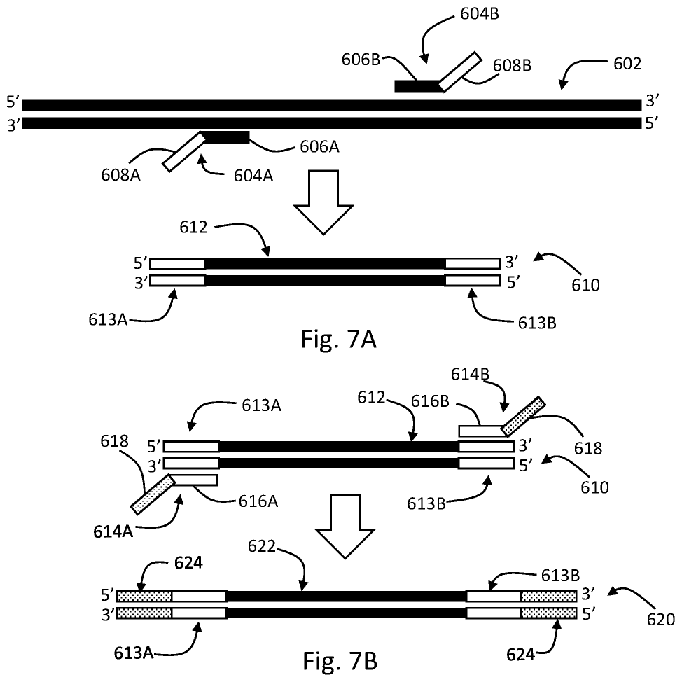

An aspect of the disclosure relates to providing an improved two-step PCR-based method that may be referred to as a dual additive PCR method, optionally for creating an amplicon library for NGS. FIG. 6 shows a flowchart illustrating blocks of a dual additive PCR method 500 in accordance with an embodiment of the disclosure, and FIGS. 7A and 7B schematically illustrate two PCR reactions comprised in the method.

As shown in FIG. 6, dual additive PCR method 500 in accordance with an embodiment of the disclosure comprises: a block 502 comprising performing a first PCR reaction using primers comprising, in a 3′ to 5′ direction, a targeting region and a universal tail overhang, such that a first PCR product of the first PCR reaction comprises an amplified region together with an added universal tail corresponding to the universal tail overhang that flanks the amplified region on both ends of the PCR product; and a block 504 comprising performing a second PCR reaction for further amplifying the amplified region comprised in the first PCR product, using primers comprising a targeting region complementary to the added universal tail. Optionally, the primers for the second PCR reaction also comprises an overhang at the 5′ end to add further components to the PCR product, such as a barcode and/or an NGS adapter.

Reference is made to FIG. 7A that schematically illustrates an embodiment of the first PCR reaction comprised in block 502. The first PCR reaction may comprise a base DNA 602 comprising a target region to be amplified, a forward primer 604A, a reverse primer 604B, and other reagents as appropriate for the PCR reaction, such as a polymerase, buffers, and the like (not shown). Forward primer 604A comprises, in a 3′ to 5′ direction, (1) a targeting region 606A complementary to a portion of base DNA 602 and (2) a forward universal tail overhang 608A. Reverse primer 604B comprises, in a 3′ to 5′ direction, (1) a targeting region 606A complementary to a portion of base DNA 602 and (2) a reverse universal tail overhang 608B. As a result, a first PCR product 610 of the first PCR reaction comprises a copy 612 of the target region of base DNA 602 flanked by a forward universal tail 613A based on the sequence of forward universal tail overhang 608A of forward primer 604A and by a reverse universal tail 613B based on the sequence of reverse universal tail overhang 608B of reverse primer 604B.

Reference is made to FIG. 7B that schematically illustrates an embodiment of the second PCR reaction comprised in block 504. The second PCR reaction may comprise first PCR product 610, a forward prime 614A, a reverse primer 614B, and other reagents as appropriate for the PCR reaction, such as a polymerase, buffers, and the like (not shown). Forward primer 614A comprises, in a 3′ to 5′ direction, (1) an targeting region 616A complementary to forward universal tail 613A comprised in first PCR product 610 and (2) a supplemental overhang 618. Reverse primer 614B comprises, in a 3′ to 5′ direction, (1) an targeting region 616B complementary to forward universal tail 613AB comprised in first PCR product 610 and (2) a supplemental overhang 618. As a result, a second PCR product 620 of the second PCR reaction comprises target region copy 612 flanked by forward universal tail 613A and reverse universal tail 613B, which is further flanked by supplemental region 624 based on supplemental overhang region 618. Depending of the uses of supplemental region 624, supplemental overhang region 618 on forward primer 614A and reverse primer 614B may be the same or different.

Supplemental overhang region 618 may comprise a barcode, by way of example embodiments of barcode 204 described herein. Additionally or alternatively, supplemental overhang region 618 may comprise an NGS adapter region having a sequence that makes supplemental region 624 of second PCR product 620 compatible with an NGS. Optionally, supplemental overhang region 618 comprises, in a 3′ to 5′ direction: (1) a barcode and (2) an NGS adapter region, such that the outermost portion of second PCR product 620, at both ends, comprise an NGS adapter region. By way of example, the NGS system for which the adapter is compatible is a commercial NGS system from, by way of example Illumina® (MiSeq® and others), Roche® (454®), and Ion-Torrent® for which compatible adapters are established and known.

In an embodiment of the disclosure, the two steps of the dual additive PCR method are performed as an std-PCR method, in which a single reaction volume simultaneously contains primers 604A and 604B for the first PCR reaction as well as primers 614A and 614B for the second PCR reaction. The first and second PCR reactions are executed sequentially by controlling the thermocycler into which the reaction volume is loaded. Optionally, a first reaction cycle operates with a first annealing temperature appropriate for targeting regions 606A and 606B of forward primer 604A and reverse primer 604B, respectively, and a second reaction cycle operates with a second annealing temperature appropriate for targeting regions 616A and 616B of forward primer 614A and reverse primer 614B, respectively. Optionally, the first annealing temperature is higher than the second annealing temperature such that the second primers 614A, 614B do not effectively anneal to their complementary sequences during the first PCR reaction cycle. Optionally, the first annealing temperature differs from the second annealing temperature by at least 8° C., between 8° C. and 15° C., between 10° C. and 14° C., or about 12° C. The first annealing temperature may be between 55° C. and 65° C., about 60° C., about 61° C. or about 62° C., and the second annealing temperature may be between 45° C. and 55° C., about 48° C., about 49° C., or about 50° C.

Optionally, base DNA 602 is a genomic DNA or a portion thereof optionally extracted from a bacterial sample. Optionally, dual additive PCR method 500, which is optionally a std-PCR method, is used in amplifying spacer regions of a CRISPR array, by way of example as block 304 of HiCRISPR method 300 as described herein. In such a case, the base DNA comprises a CRISPR array of a microbe or a portion thereof, comprising a plurality of DR and spacer regions, shown by way of example in FIGS. 1A and 1B. As such, targeting regions 606A and 606B may be complementary to portions of direct repeat (DR) regions within a CRISPR array of the microbe. By way of example, targeting region 606A may comprise a DRa sequence, and targeting region 606B may comprise a DRb sequence, for amplifying spacer-comprising fragments from a M. tuberculosis CRISPR array. Moreover, supplemental overhang region 618 may comprise, in a 3′ to 5′ direction, a well-specific barcode and an NGS adapter region, so that second PCR product 620 comprises a well-specific barcode and an NGS adapter compatible with an NGS system. It will be appreciated that second PCR product 620 made with such an overhang region 618 would be generated with the NGS adapter during the PCR process, and so would not require a separate ligation step to add the NGS adapter. PCR product 620 from a plurality of dual additive PCR reactions, each with genomic DNA isolated from different bacterial samples, can be pooled to form a library (block 306), sequenced with NGS (block 308).

### EXAMPLE 3

A HiCRISPR method in accordance with an embodiment of the disclosure was carried out with microbial DNA isolates taken from 96 M. tuberculosis samples. The PCR method used was a dual expansion PCR method in accordance with an embodiment of the disclosure. Ninety six (96) first PCR reactions 502 and ninety six second PCR reactions 504 were performed to amplify spacers from each of the M. tuberculosis DNA isolates.

Each first PCR reaction (block 502) comprised one of the 96 M. tuberculosis DNA isolates and the following 8 primers as shown in Table 2:

TABLE 2

| | | | | SEQ |
|---|---|---|---|---|
| | Direc- | | | ID |
| Name | tion | Sequence (5′ -> 3′) | length | NO |
| UT- | forward | TCGTCGGCAGCGTCAGATGTGTATAAGA | 51 | 201 |
| DRa1 | | GACAGGGTTTTGGGTCTGACGAC | | |
| UT- | forward | TCGTCGGCAGCGTCAGATGTGTATAAGA | 52 | 202 |
| DRa2 | | GACAGNGGTTTTGGGTCTGACGAC | | |
| UT- | forward | TCGTCGGCAGCGTCAGATGTGTATAAGA | 53 | 203 |
| DRa3 | | GACAGNNGGTTTTGGGTCTGACGAC | | |
| UT- | forward | TCGTCGGCAGCGTCAGATGTGTATAAGA | 54 | 204 |
| DRa4 | | GACAGNNNGGTTTTGGGTCTGACGAC | | |
| UT- | Reverse | GTCTCGTGGGCTCGGAGATGTGTATAAG | 52 | 205 |
| DRb1 | | AGACAGCCGAGAGGGGACGGAAAC | | |
| UT- | Reverse | GTCTCGTGGGCTCGGAGATGTGTATAAG | 53 | 206 |
| DRb2 | | AGACAGNCCGAGAGGGGACGGAAAC | | |
| UT- | Reverse | GTCTCGTGGGCTCGGAGATGTGTATAAG | 54 | 207 |
| DRb3 | | AGACAGNNCCGAGAGGGGACGGAAAC | | |
| UT- | Reverse | GTCTCGTGGGCTCGGAGATGTGTATAAG | 55 | 208 |
| DRb4 | | AGACAGNNNCCGAGAGGGGACGGAAAC | | |

As shown in Table 2, each of the first PCR reactions employed four forward primers, UT-DRa1, UT-DRa2, UT-DRa4, and UT-DRa4. Each of these forward primers comprised, in a 5′ to 3′ direction, a forward universal overhang (TCGTCGGCAGCGTC; SEQ ID NO: 209); a 19 base-pair (bp) Mosaic End overhang (AGATGTGTATAAGAGACAG; SEQ ID NO: 210) that serves as a sequencing primer binding site and a transposase recognition sequence, and a DRa targeting region (GGTTTTGGGTCTGACGAC; SEQ ID:3). Each of the four forward primers comprised between 0 and 3 nucleotides (“N”) preceding the DRa targeting region, which provides improved complexity of the resulting PCR products for improved sequencing performance in the NGS.

Also as shown in Table 2, each of the first PCR reactions employed four reverse primers, UT-DRb1, UT-DRb2, UT-DRb3, UT-DRb4, each of which comprised, in a 5′ to 3′ direction, a reverse universal tail overhang (GTCTCGTGGGCTCGG; SEQ ID NO: 211); a 19 bp Mosaic End overhang (AGATGTGTATAAGAGACAG; SEQ ID NO: 210), and a DRb targeting region (CCGAGAGGGGACGGAAAC; SEQ ID NO: 4). As with the forward primers, each of the four reverse primers comprised between 0 and 3 nucleotides (“N”) preceding the DRb targeting region. The first PCR reactions were performed in a thermocycler programmed to consist of the following steps:
a) initiation step: 95° C. for 15 minutes;
b) second initiation step: 96° C. for 3 minutes;
c) denaturation step: 96° C. for 60 seconds;
d) annealing step: 55° C. for 60 seconds;
e) elongation step: 72° C. for 30 seconds;
f) repeat steps c-e for 40 cycles; and
g) final elongation step: 72° C. for 5 minutes.

Each second PCR reaction (block 504) comprised one of the 96 M. tuberculosis DNA isolates, as well as one forward primer and one reverse primer selected from the following primers as shown in Table 3:

TABLE 3

| | | | | SEQ |
|---|---|---|---|---|
| | Direc- | | | ID |
| Name | tion | Sequence (5′ -> 3′) | Length | NO |
| proMID- | for- | AATGATACGGCGACCACCGAGATC | 54 | 212 |
| 1F | ward | TACACGGAGTTAGGACTCGTCGGC | | |
| | | AGCGTC | | |
| proMID- | for- | AATGATACGGCGACCACCGAGATC | 54 | 213 |
| 2F | ward | TACACCCTCAATCCTGTCGTCGGC | | |
| | | AGCGTC | | |
| proMID- | for- | AATGATACGGCGACCACCGAGATC | 54 | 214 |
| 3F | ward | TACACTTCTGGCTTCATCGTCGGC | | |
| | | AGCGTC | | |
| proMID- | for- | AATGATACGGCGACCACCGAGATC | 54 | 215 |
| 4F | ward | TACACAAGACCGAAGTTCGTCGGC | | |
| | | AGCGTC | | |
| proMID- | for- | AATGATACGGCGACCACCGAGATC | 54 | 216 |
| 5F | ward | TACACTGTCCACGTTGTCGTCGGC | | |
| | | AGCGTC | | |
| proMID- | for- | AATGATACGGCGACCACCGAGATC | 54 | 217 |
| 6F | ward | TACACACAGGTGCAACTCGTCGGC | | |
| | | AGCGTC | | |
| proMID- | for- | AATGATACGGCGACCACCGAGATC | 54 | 218 |
| 7F | ward | TACACGTGAACATGGTTCGTCGGC | | |
| | | AGCGTC | | |
| proMID- | for- | AATGATACGGCGACCACCGAGATC | 54 | 219 |
| 8F | ward | TACACCACTTGTACCATCGTCGGC | | |
| | | AGCGTC | | |
| proMID- | for- | AATGATACGGCGACCACCGAGATC | 54 | 220 |
| 9F | ward | TACACTGCAGCATATTTCGTCGGC | | |
| | | AGCGTC | | |
| proMID- | for- | AATGATACGGCGACCACCGAGATC | 54 | 221 |
| 10F | ward | TACACACGTCGTATAATCGTCGGC | | |
| | | AGCGTC | | |
| proMID- | for- | AATGATACGGCGACCACCGAGATC | 54 | 222 |
| 11F | ward | TACACGTAATACGCGGTCGTCGGC | | |
| | | AGCGTC | | |
| proMID- | for- | AATGATACGGCGACCACCGAGATC | 54 | 223 |
| 12F | ward | TACACTTACTGTAGGTTCGTCGGC | | |
| | | AGCGTC | | |
| proMID- | Re- | CAAGCAGAAGACGGCATACGAGAT | 50 | 224 |
| 13R | verse | AATGACATCCAGTCTCGTGGGCTCGG | | |
| proMID- | Re- | CAAGCAGAAGACGGCATACGAGAT | 50 | 225 |
| 14R | verse | GGCAGTTCTTGGTCTCGTGGGCTCGG | | |
| proMID- | Re- | CAAGCAGAAGACGGCATACGAGAT | 50 | 226 |
| 15R | verse | CAGTCACGAACGTCTCGTGGGCTCGG | | |
| proMID- | Re- | CAAGCAGAAGACGGCATACGAGAT | 50 | 227 |
| 16R | verse | CGTAACCGCATGTCTCGTGGGCTCGG | | |
| proMID- | Re- | CAAGCAGAAGACGGCATACGAGAT | 50 | 228 |
| 17R | verse | GCATTGGCGTAGTCTCGTGGGCTCGG | | |
| proMID- | Re- | CAAGCAGAAGACGGCATACGAGAT | 50 | 229 |
| 18R | verse | TACGGTTATGCGTCTCGTGGGCTCGG | | |
| proMID- | Re- | CAAGCAGAAGACGGCATACGAGAT | 50 | 230 |
| 19R | verse | GGTTCCAATTAGTCTCGTGGGCTCGG | | |
| proMID- | Re- | CAAGCAGAAGACGGCATACGAGAT | 50 | 231 |
| 20R | verse | TTGGAACCAGCGTCTCGTGGGCTCGG | | |

Each of the 12 above-listed forward primers for the second PCR reaction comprised, in a 5′ to 3′ direction, a P5 universal adapter overhang (AATGATACGGCGACCACCGAGATCTACAC; SEQ ID NO: 232) for introducing a P5 universal adapter to make the PCR product compatible for NGS flow cell binding; an 11-nucleotide ProMID barcode overhang (selected from SEQ ID NOS: 9-104) for introducing a barcode, and a forward universal tail targeting region (TCGTCGGCAGCGTC; SEQ ID NO: 209). Each of the 8 above-listed reverse primers for the second PCR reaction comprised, in a 5′ to 3′ direction, a P7 universal adapter overhang (CAAGCAGAAGACGGCATACGAGAT; SEQ ID NO: 233) for introducing a P7 universal adapter to make the PCR product compatible for NGS flow cell binding; an 11-nucleotide ProMID barcode over (selected from SEQ ID NOS: 9-104) not used in any of the 12 forward primers for introducing another barcode, and a reverse universal tail targeting region (GTCTCGTGGGCTCGG; SEQ ID NO: 211)). For each of the 96 reactions, a different combination of one of the 12 forward primers and one of the 8 reverse primers were used so that each reaction was indexed with one of 96 (12×8) unique combinations of one forward primer and one reverse primer. The second PCR reactions were performed in a thermocycler programmed to consist of the following steps:
a) initiation step: 95° C. for 15 minutes;
b) second initiation step: 96° C. for 3 minutes;
c) denaturation step: 96° C. for 60 seconds;
d) annealing step: 58° C. for 60 seconds;
e) elongation step: 72° C. for 30 seconds;
f) repeat steps c-e for 15 cycles; and
g) final elongation step: 72° C. for 5 minutes.

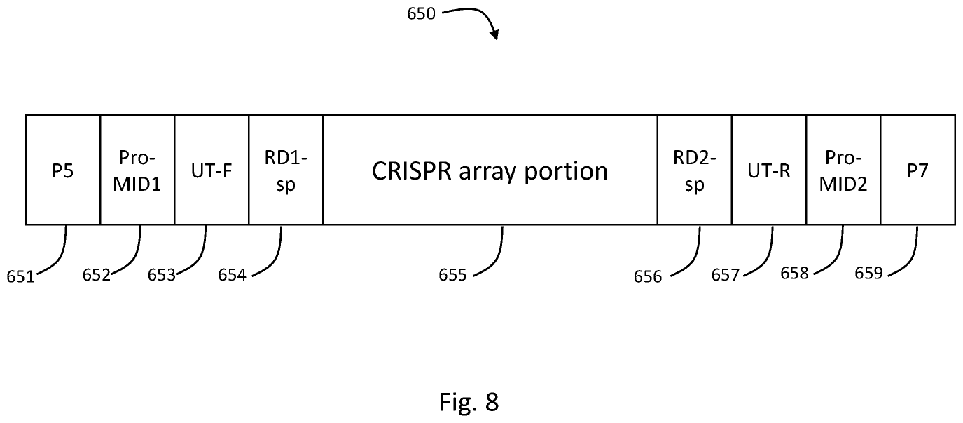

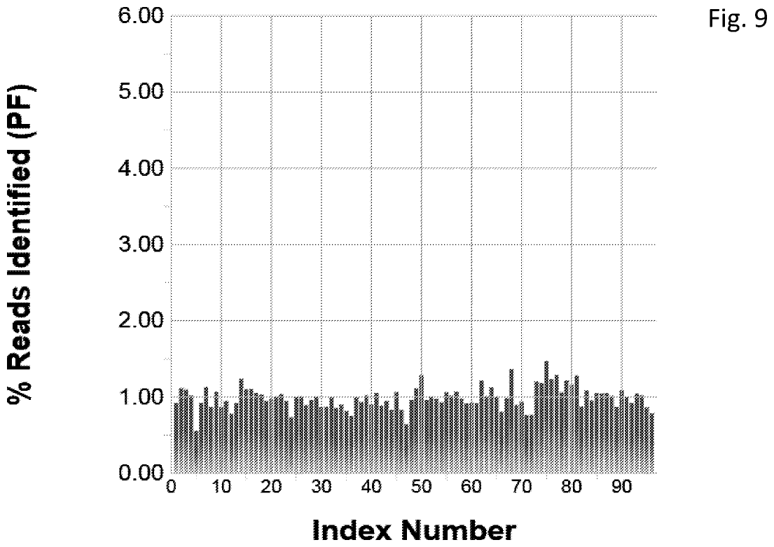

Reference is made to FIG. 8, which schematically shows an example second PCR product 650 of the second PCR reaction described hereinabove, which includes all elements needed to sequence the amplified portions of the CRISPR array in a MiSeq® system (Illumina®). PCR product 650, which is a double-stranded DNA molecule, included the following regions: a P5 universal adapter region 651, a first Pro-MID (Pro-MID1) region 652, a forward universal tail (UT-F) region 653, a RD1-sp (Read 1 Sequence primer binding) region 654; an amplified portion of a CRISPR array 655 comprising at least one spacer and a portion of the flanking DR regions; a RD2-sp region 656, a reverse universal tail (UT-R) region 657, a second Pro-MID (Pro-MID2) region 658, and a P7 universal adapter region 659. The second PCR products 650 from each of the 96 samples were then be pooled, purified with a QIAquick® PCR purification kit (Qiagen®) and sequenced with a MiSeq® NGS system (Illumina®). Sequencing quality was high, with an average % Q30 score of 88.51, a cluster density of 989 K/mm², and a yield of 1,245,614 reads. The NGS sequence data was analyzed to demultiplex the sequence data according to barcodes so that sequence data portions sharing a same barcode pair, and thus generated in the same reaction well from the same microbial DNA sample, were sorted together. FIG. 9 shows a % abundance of reads characterized by a given barcode pair (and thus known to be derived from a given reaction well) for all 96 samples. It was found that the read distribution of the 96 samples was relatively even, with each sample representing between about 0.5% and about 1.4% of the total reads (if distribution was perfectly equal between the 96 samples, each sample would be 1.04% of the total reads). This distribution was notably achieved without a normalization step, which is typically included in GNS library preparation.

Each of the 96 samples were samples of M. tuberculosis strain H37Rv. The reads from some individual samples were mapped against the H37Rv genome using Bowtie2®, and it was found that substantially all of the reads mapped onto the CRISPR/Cas locus. Moreover, the reads identified additional spacers that are not included in the international scheme of spacers for this strain, thus highlighting an advantage of sequencing-based SPACERome analysis, which is not dependent on a canonical set of spacers, over spoligotyping.

### EXAMPLE 4

While the two-step dual additive PCR (as described in Example 3) was successfully applied in generating a CRISPR library for NGS and high-throughput generation of SPACERome, the process can be made even more efficient if the two PCR reactions can be performed as a std-PCR in a single reaction volume. To make std-PCR possible, the DRa and DRb targeting sequences (corresponding to targeting regions 606A and 606B as shown in FIG. 7A) in the set of primers (corresponding to primers 604A and 604B as shown in FIG. 7A) for the first PCR reaction were redesigned in order to make the respective annealing temperatures (Tm) higher and more uniform. In addition, the targeting regions (corresponding to targeting regions 616A and 616B as shown in FIG. 7B) in the set of primers (corresponding to primers 614A and 614B as shown in FIG. 8b) for the second PCR reaction, which are complementary to the forward and reverse universal tails in first PCR produce 610, were redesigned in order to make the respective annealing temperatures lower.

Table 4 below provides the sequences of the DRa and DRb targeting regions together with the respective modified version, mDRa and mDRb:

TABLE 4

| | | | Tm | SEQ |
|---|---|---|---|---|
| | | | (deg. | ID |
| name | sequence (5′ -> 3′) | length | C.) | NO |
| DRa | GGTTTTGGGTCTGACGAC | 18 | 59 | 3 |
| mDRa | GGGTTTTGGGTCTGACGAC | 19 | 62 | 234 |
| DRb | CCGAGAGGGGACGGAAAC | 18 | 63 | 4 |
| mDRb | CGAGAGGGGACGGAAAC | 17 | 61 | 235 |

The DRa sequence has a length of 18 nucleotides and a Tm of 59° C., while the DRb sequence has a length of 18 nucleotides and a Tm of 63° C. Each sequence was modified to reduce the difference between the respective Tm's: the DRa sequence was modified to the mDRa sequence by adding an additional guanine on the 5′ end, thus increasing the Tm from 59° C. to 62° C. while still allowing for complete annealing of the targeting region to the template DNA; and the DRb sequence was modified to the mDRb sequence by removing the cysteine at the 5′ end, thus reducing the Tm from 63° C. to 61° C. while still allowing for complete annealing of the targeting region to the template DNA.

Table 5 below provides the sequences of the modified universal tail targeting regions, which may by way of example be comprised in primers used in a second PCR reaction 504 of std-PCR.

TABLE 5

| | | | Tm | SEQ |
|---|---|---|---|---|
| | | | (deg. | ID |
| name | sequence (5′ -> 3′) | length | C.) | NO |
| UT-F | TCGTCGGCAGCGTC | 14 | 60 | 209 |
| sUT-F | TCGTCGGCAGC | 11 | 50 | 236 |
| UT-R | GTCTCGTGGGCTCGG | 15 | 60 | 211 |
| sUT-R | GTCTCGTGGGCT | 12 | 49 | 237 |

Both the forward universal tail (UT-F) and the reverse universal tail (UT-R) have a Tm of 60° C. Each sequence was modified to reduce the Tm: the UT-F sequence was modified to the sUT-F sequence by removing the three nucleotides at the 3′ end, thus reducing the Tm to 50° C.; and the UT-R sequence was modified to the sUT-R sequence by removing the three nucleotides at the 3′ end, thus reducing the Tm to 49° C.

A HiCRISPR method in accordance with an embodiment of the disclosure was carried out with samples from microbial DNA isolates taken from 96 M. tuberculosis samples. The PCR amplification step (corresponding to block 304 of method 300 as shown in FIG. 3) employed an embodiment of std-PCR method, with a first PCR reaction (corresponding to block 502 of method 500 as shown in FIG. 6) using primers comprising the mDRa, mDRb sequences as targeting regions and a second PCR reaction (corresponding to block 504 of method 500 as shown in FIG. 6) using primers comprising the sUT-F and sUT-R sequences as targeting regions.

Each PCR reaction volume comprised one of the 96 M. tuberculosis DNA isolates and each of the following 8 primers (4 forward primers and 4 reverse primers) as shown in Table 6 for the first PCR reaction 502:

TABLE 6

| | | | | SEQ |
|---|---|---|---|---|
| | Direc- | | | ID |
| Name | tion | Sequence (5′ -> 3′) | Length | NO |
| UT- | for- | TCGTCGGCAGCGTCAGATGTGTATAAGA | 53 | 238 |
| mDRa1 | ward | GACAGGGGTTTTGGGTCTGACGAC | | |
| UT- | for- | TCGTCGGCAGCGTCAGATGTGTATAAGA | 54 | 239 |
| mDRa2 | ward | GACAGNGGGTTTTGGGTCTGACGAC | | |
| UT- | for- | TCGTCGGCAGCGTCAGATGTGTATAAGA | 55 | 240 |
| mDRa3 | ward | GACAGNNGGGTTTTGGGTCTGACGAC | | |
| UT- | for- | TCGTCGGCAGCGTCAGATGTGTATAAGA | 56 | 241 |
| mDRa4 | ward | GACAGNNNGGGTTTTGGGTCTGACGAC | | |
| UT- | Re- | GTCTCGTGGGCTCGGAGATGTGTATAAG | 51 | 242 |
| mDRb1 | verse | AGACAGCGAGAGGGGACGGAAAC | | |
| UT- | Re- | GTCTCGTGGGCTCGGAGATGTGTATAAG | 52 | 243 |
| mDRb2 | verse | AGACAGNCGAGAGGGGACGGAAAC | | |
| UT- | Re- | GTCTCGTGGGCTCGGAGATGTGTATAAG | 53 | 244 |
| mDRb3 | verse | AGACAGNNCGAGAGGGGACGGAAAC | | |
| UT- | Re- | GTCTCGTGGGCTCGGAGATGTGTATAAG | 54 | 245 |
| mDRb4 | verse | AGACAGNNNCGAGAGGGGACGGAAAC | | |

As shown in Table 6, each of first PCR reactions 502 employed four forward primers, UT-mDRa1, UT-mDRa2, UT-mDRa4, and UT-mDRa4. Each of these forward primers comprised, in a 5′ to 3′ direction, a forward universal tail overhang (TCGTCGGCAGCGTC; SEQ ID NO: 209); a 19 base-pair (bp) Mosaic End overhang (AGATGTGTATAAGAGACAG; SEQ ID NO: 210) that serves as a sequencing primer binding site and a transposase recognition sequence, and a mDRa targeting region (GGGTTTTGGGTCTGACGAC; SEQ ID NO: 235). Each of the four forward primers comprised between 0 and 3 nucleotides (“N”) preceding the mDRa targeting region, which provides improved complexity of the resulting PCR products for improved sequencing performance in the NGS.

Also as shown in Table 6, each of the first PCR reactions employed four reverse primers, UT-mDRb1, UT-mDRb2, UT-mDRb3, UT-mDRb4, each of which comprised, in a 5′ to 3′ direction, a reverse universal tail overhang (GTCTCGTGGGCTCGG; SEQ ID NO: 211); a 19 bp Mosaic End overhang (AGATGTGTATAAGAGACAG; SEQ ID NO: 210), and a mDRb targeting region (CGAGAGGGGACGGAAAC; SEQ ID NO: 235). As with the forward primers, each of the four reverse primers comprised between 0 and 3 nucleotides (“N”) preceding the DRb targeting region.

Each of the 96 reaction volumes also included a unique combination of one forward primer and one reverse primer selected from the following primers as shown in Table 7, for the second PCR reaction:

TABLE 7

| | Di- | | | SEQ |
|---|---|---|---|---|
| | rec- | | | ID |
| Name | tion | Sequence (5′ -> 3′) | Length | NO |
| 1Fnew | for- | AATGATACGGCGACCACCGAGATC | 48 | 246 |
| | ward | TACACAAGCAGGATCGTCGGCAGC | | |
| 2Fnew | for- | AATGATACGGCGACCACCGAGATC | 48 | 247 |
| | ward | TACACTTCGTCCTTCGTCGGCAGC | | |
| 3Fnew | for- | AATGATACGGCGACCACCGAGATC | 48 | 248 |
| | ward | TACACCCTACTTCTCGTCGGCAGC | | |
| 4Fnew | for- | AATGATACGGCGACCACCGAGATC | 48 | 249 |
| | ward | TACACGGATGAAGTCGTCGGCAGC | | |
| 5Fnew | for- | AATGATACGGCGACCACCGAGATC | 48 | 250 |
| | ward | TACACAACAGCAATCGTCGGCAGC | | |
| 6Fnew | for- | AATGATACGGCGACCACCGAGATC | 48 | 251 |
| | ward | TACACTTGTCGTTTCGTCGGCAGC | | |
| 7Fnew | for- | AATGATACGGCGACCACCGAGATC | 48 | 252 |
| | ward | TACACGTTGATGGTCGTCGGCAGC | | |
| 8Fnew | for- | AATGATACGGCGACCACCGAGATC | 48 | 253 |
| | ward | TACACAGAGTGTTTCGTCGGCAGC | | |
| 9Fnew | for- | AATGATACGGCGACCACCGAGATC | 48 | 254 |
| | ward | TACACTCTCACAATCGTCGGCAGC | | |
| 10Fnew | for- | AATGATACGGCGACCACCGAGATC | 48 | 255 |
| | ward | TACACAAGACACCTCGTCGGCAGC | | |
| 11Fnew | for- | AATGATACGGCGACCACCGAGATC | 48 | 256 |
| | ward | TACACCAGGTAGTTCGTCGGCAGC | | |
| 12Fnew | for- | AATGATACGGCGACCACCGAGATC | 48 | 257 |
| | ward | TACACACTTGCTGTCGTCGGCAGC | | |
| 13Rev_new | Re- | CAAGCAGAAGACGGCATACGAGAT | 44 | 258 |
| | verse | GTCGTTCAGTCTCGTGGGCT | | |
| 14Rev_new | Re- | CAAGCAGAAGACGGCATACGAGAT | 44 | 259 |
| | verse | ATCTGGACGTCTCGTGGGCT | | |
| 15Rev_new | Re- | CAAGCAGAAGACGGCATACGAGAT | 44 | 260 |
| | verse | GTATAGCGGTCTCGTGGGCT | | |
| 16Rev_new | Re- | CAAGCAGAAGACGGCATACGAGAT | 44 | 261 |
| | verse | CATATCGCGTCTCGTGGGCT | | |
| 17Rev_new | Re- | CAAGCAGAAGACGGCATACGAGAT | 44 | 262 |
| | verse | TATGCGACGTCTCGTGGGCT | | |
| 18Rev_new | Re- | CAAGCAGAAGACGGCATACGAGAT | 44 | 263 |
| | verse | ATGATACCGTCTCGTGGGCT | | |
| 19Rev_new | Re- | CAAGCAGAAGACGGCATACGAGAT | 44 | 264 |
| | verse | TACTATGGGTCTCGTGGGCT | | |
| 20Rev_new | Re- | CAAGCAGAAGACGGCATACGAGAT | 44 | 265 |
| | verse | AGTGGTAGGTCTCGTGGGCT | | |

Each of the 12 above-listed forward primers for the second PCR reaction comprised, in a 5′ to 3′ direction, a P5 universal adapter overhang (AATGATACGGCGACCACCGAGATCTACAC; SEQ ID NO: 232); an 8-nucleotide ProMID barcode overhang (selected from SEQ ID NOS: 105-200), and a sUT-F targeting region (TCGTCGGCAGC; SEQ ID NO: 236). Each of the 8 above-listed reverse primers for the second PCR reaction comprised, in a 5′ to 3′ direction, a P7 universal adapter overhang (CAAGCAGAAGACGGCATACGAGAT; SEQ ID NO: 233); an 8-nucleotide ProMID barcode (selected from SEQ ID NOS: 105-200) not used in any of the 12 forward primers, and a sUT-R targeting region (GTCTCGTGGGCT; SEQ ID NO: 237). For each of the 96 reactions, a different combination of one of the 12 forward primers and one of the 8 reverse primers were used so that each reaction was indexed with one of 96 (12×8) unique combinations of one forward primer and one reverse primer.

The 96 std-PCR reactions were performed in a thermocycler programmed to consist of the following steps, which included the first PCR reaction (steps b-e) and the second PCR reaction (steps f-i) being performed in sequence in the same reaction volume:
a) initiation step: 95° C. for 2 minutes;
b) denaturation step: 95° C. for 20 seconds;
c) annealing step: 61° C. for 10 seconds;
d) elongation step: 70° C. for 10 seconds;
e) repeat steps b-d for 30 cycles;
f) denaturation step: 95° C. for 30 seconds;
g) annealing step: 49° C. for 10 seconds;
h) elongation step: 70° C. for 10 seconds; and
i) repeat steps f-h for 10 cycles.

The final PCR product for the std-PCR method, as with the two-step dual additive PCR method, is ready for NGS without further modifications. As such, the final PCR products were pooled, purified with magnetic beads and sequenced with a MiSeq® NGS system (Illumina®). Sequencing quality was high, with an average % Q30 score of 92.48. Each of 96 samples were samples of different M. tuberculosis clinical isolates and controls. The reads from some individual samples were mapped against the H37Rv genome using Bowtie2®, and it was found that substantially all of the reads mapped onto the CRISPR/Cas locus of the H37Rv genome.

While the examples above were conducted with samples of M. tuberculosis, the procedure determining a SPACERome would be identical for any other bacterial sample provided that a different DR targeting sequence is used in the primers for the first PCR reaction. By way of example, the DR region sequence is known for at least the following bacterial species: Lactobacillus gasseri, Bifidobacterium spp., Erwinia amylovora, E. coli, Salmonella enterica, Campylobacter spp., Acinetobacter baumannii, Group A Streptococcus, Group B Streptococcos, Cronobacter sakazakii, Yersinia species, and Mycobacterium tuberculosis.

It will be appreciated that a dual additive PCR method in accordance with an embodiment of the disclosure (whether in the two-step or std-PCR form) can be applied to any high-throughput genetic analysis, in combination determining a SPACERome of a microbial sample, or on its own. By way of example, genes that serve as a marker for lineage marker, antibiotic resistance or virulence may be amplified and made ready for NGS provided that first PCR reaction 502 includes a forward primer for amplifying the appropriate gene comprising a forward universal tail region situated 3′ of the targeting region, and a corresponding reverse primer comprising a reverse universal tail region situated 3′ of the targeting region. It will also be appreciated that a dual additive PCR method in accordance with an embodiment of the disclosure can be applied to a wide variety of applications beyond CRISPR-typing, where high-throughput genetic analysis of biological samples, optionally using NGS, is useful. Examples of application for a dual additive PCR method in accordance with an embodiment of the disclosure include: validating identity of natural products (including wild-harvested fish and plants); tracking genetic identity of agricultural products; epidemiologically tracking clinically relevant microbial species and/or strains (for example in a hospital, a city, or a country); identifying genomic factors contributing to tumorigenesis; and gut microbiome characterization of subjects.

An aspect of the disclosure relates to providing a method of phylogenetic analysis of a bacterial sample based on a SPACERome of the bacterial sample. Phylogenetic analysis may comprise generating a phylogenetic tree, a branching diagram showing evolutionary relationships among various biological species, or strains within species. A “gold-standard” method of generating phylogenetic tree is a Codon Tree method which uses amino acid and nucleotide sequences from a defined number of picked randomly genes in a genome to build an alignment and then generate a tree based on the differences within those selected sequences. However, the Codon Tree method typically uses a whole genome sequence (WGS) which is time-consuming and expensive to prepare. In addition, to achieve sufficient resolution for phylogenetic relationships between closely related species or strains within a species using the Codon Tree method, analysis of 1000 or more genes may be required.

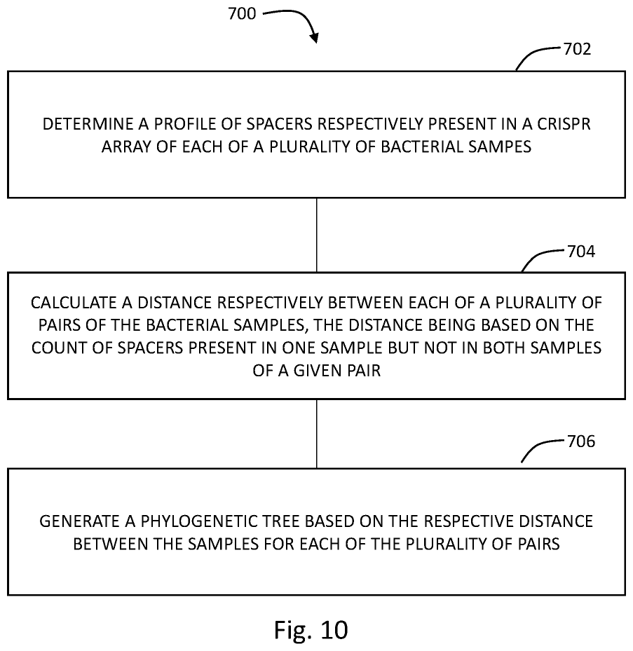

FIG. 10 shows a flowchart 700 illustrating steps of a SPACERome phylogeny method in accordance with an embodiment of the disclosure. As shown in FIG. 10, SPACERome phylogeny method 700 in accordance with an embodiment of the disclosure comprises: a block 702 comprising determining a profile of spacers respectively present in a CRISPR array of each of a plurality of bacterial samples; and a block 704 comprising calculating a distance respectively between each of a plurality of pairs of bacterial samples, each pair being a unique pair selected from the plurality of bacterial samples, wherein, for each unique pair, the distance is based on the count of spacers present in one sample but not in both samples of the unique pair; and a block 706 comprising generating a phylogenetic tree for the plurality of bacterial samples based on the respective distance between the samples for each pair of the plurality of pairs.

Reference is made to block 702. The profile of spacers may be determined by any method known in the art, by way of example an embodiment of a HiCRISPR method as disclosed herein, a spoligotyping method, or analyzing a WGS of a given bacterial sample.

Reference is made to block 704. The distance between samples of a given pair of samples is based on the sum X+Y of the count X of spacers present in a first sample of the pair but not in a second sample of the pair and the count Y of spacers present in the second sample but not in the first sample. By way of example, a given microbial species may have one or more of 10 spacers labeled as: a, b, c, d, e, f, g, h, i, and j. If the first sample is determined to have spacers a, b, e, and g and the second sample is determined to have spacers a, b, h, i, and j, the sum X+Y for the pair is calculated to be 2+3=5, because the first sample has 2 spacers, e and g, which are not present in the second sample, and the second sample has 3 spacers, h, i, and j, which are not present in the first sample.

Regarding block 706, the phylogenetic tree is generated as a neighbor joining tree based on the respective distances determined for each of the pairs.

### EXAMPLE 5

A WGS was obtained for each of 30 clones of presumed Cronobacter sakazakii (C. sakazakii) isolated from Powdered Infant Formula (PIF) or clinical samples. The bacterial species of these 30 closes were determined to be C. sakazakii based on matrix-assisted laser desorption/ionization time-of-flight (MALDI-TOF) mass spectroscopy. The WGSs were obtained with NGS using MiSeq® Reagent Kit v2 (500-cycles). As an out-group control, WGS of a Cronobacter muytjensii (C. muytjensii) strain (ATCC-51329) obtained from American Type Culture Collection (ATCC) was also included in the analysis.

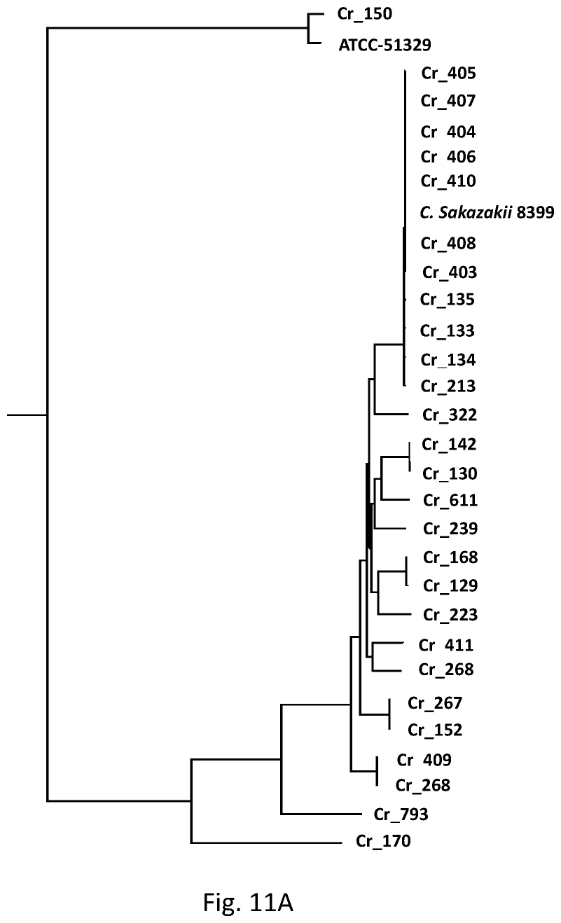

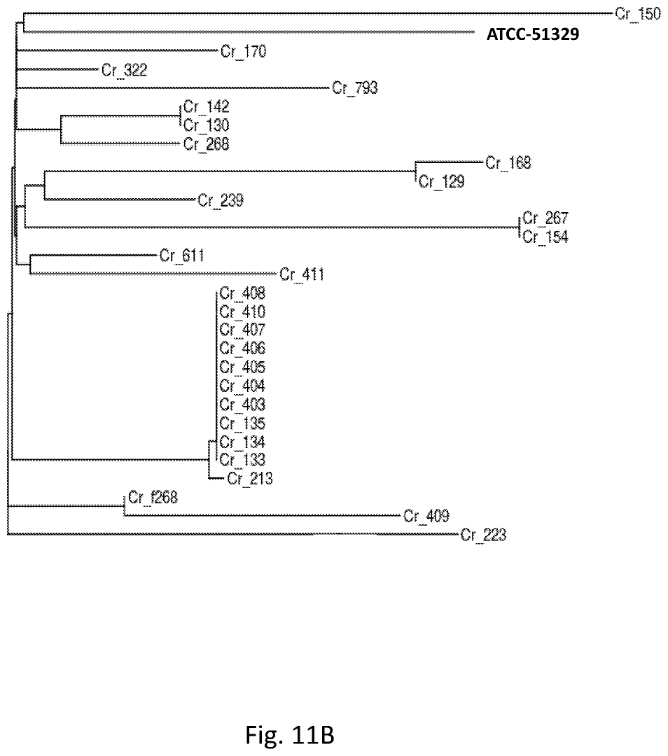

FIG. 11A shows a “gold-standard” phylogenetic tree generated from the WGS sequences from the 31 samples, based on a Codon Tree method. The phylogenetic tree shown in FIG. 11A was generated with a Codon Tree phylogeny module available at www.patricbrc.org. Because the genomes analyzed were from closely related strains, the tree was generated based on aligning 1000 genes common to the samples. The tree shown in FIG. 11A was based on alignment of 417,501 amino acids and 1,252,503 nucleotides, and was generated after a total job duration of 3.65 hours.

A comprehensive genome analysis of Cr_150 and Cr_170 revealed that the MALDI-TOF identification was incorrect: the Cr_150 sample was in fact a strain of C. muytjensii, and Cr_170 was in fact a strain of Cronobacter turicensis (C. turicensis). As can be seen in FIG. 11A, the Codon Tree phylogeny method was able to resolve the difference between C. sakazakii, C. muytjensii and C. turicensis: Cr_150 and ATCC-51329, which is also a C. muytjensii strain, were clustered with each other but not with the rest of the samples, and Cr_170 was a lone sample that was not clustered with any of the C. sakazakii or C. muytjensii samples.

Reference is made to FIG. 11B, which shows a SPACERome-based phylogenetic tree in accordance with an embodiment of the disclosure. The spacers present in each of the 31 samples were determined from the WGS using sequence analysis tools available in the Pathosystems Resource Integration Center (PATRIC) web site at www.patricbrc.org. There are 31(31-1)/2=465 possible sample pairs in the group of 31 samples. For each pair, a distance between the two samples in the pair was determined based on the sum of the count of spacers extracted from the NGS sequence of the first sample that were not extracted from the NGS sequence of the second sample and the count of spacers extracted from the NGS sequence of the second sample that were not extracted from the NGS sequence of the first sample. FIG. 11B shows a phylogenetic tree of the 31 samples generated as a neighbor joining tree based on the respective distances determined for each of the pairs.

It will be appreciated that the SPACERome-based phylogenetic tree is remarkably similar to the tree generated with the gold-standard Codon tree method. By way of example, the SPACERome-based tree shows a large cluster including samples Cr_133, Cr_134, Cr_135, Cr_403, Cr_404, Cr_405, Cr_406, Cr_407, Cr_408 and Cr_410. The “gold-standard” method also shows a large cluster including all of the samples (Cr_133, Cr_134, Cr_135, Cr_403, Cr_404, Cr_405, Cr_406, Cr_407, Cr_408, and Cr_410). The only difference is Cr_213, which is included in the same cluster in the “gold-standard” method and is placed in an immediately adjacent cluster as a sole entry in the CRISPR-based method. In addition, both methods place Cr_142 and Cr_130 in a same clustser, and place Cr_129 and Cr_168 in a same cluster. Moreover, can be seen in FIG. 11B, the SPACERome phylogeny method was able to resolve the difference between C. sakazakii, C. muytjensii and C. turicensis: Cr_150 and ATCC-51329, which is also a C. muytjensii strain, were clustered with each other but not with the rest of the samples, and Cr_170 was a lone sample that was not clustered with any of the C. sakazakii or C. muytjensii samples.

It will be appreciated, based on the SPACERome phylogeny results shown in FIG. 11B, that the SPACERome not only provides a signature for strains within a species, but a signature for a given species.

In the results shown in FIGS. 11A-B, the SPACERome of the Cronobacter samples were generated from sequence analysis of the samples' respective WGS. However, the same SPACERome can be generated more quickly and inexpensively with an embodiment of a HiCRIPSR method as disclosed herein, which can then be used to generate a SPACERome-based phylogenetic tree in accordance with an embodiment of the disclosure, which as shown in FIG. 12B is comparable to a tree created using a “gold-standard” Codon Tree method in terms of sensitivity and accuracy. Thus, the combination of SPACERome determination with a HiCRISPR method and SPACERome-based phylogeny provides an unprecedently cheap, quick, and convenient way to perform phylogenetic analysis of bacterial samples. Whereas this particular example was performed with Cronobacter samples, it will be appreciated that a similar CRISPRome-based phylogenetic analysis may be performed using the method described in herein with any other bacterial species comprising a CRISPR array.

There is therefore provided in accordance with an embodiment of the disclosure a set of unique DNA barcodes characterized by at least four of the following criteria: No barcode comprises repeated nucleotides of more than 2 nucleotides in length; Each barcode has a GC content for between 30% and 60%; For any pair of barcodes selected from the set, the minimum number of nucleotide positions where the respective nucleotides are not the same is 4 nucleotides; For any pair of barcodes selected from the set, the maximum length of a common sub-sequence shared by the pair is 8 nucleotides; A length of a palindrome within a barcode is at most 9 nucleotides; and Maximum length of a reverse complementary sub-sequence between any pair of barcodes is 8 nucleotides. Optionally, the set of unique DNA barcodes is characterized by all of the above criteria. Optionally, the set of unique DNA barcodes is further characterized by at least one of the following criteria: the GC content of for each barcode is between 35% and 55%; less than 10% of barcodes in the set of unique barcodes comprise a palindrome of 6 or more nucleotides in length; the maximum length of a palindrome comprised in a barcode is 6 nucleotides; and less than 2% of pairs of barcodes out of all possible combinations of barcode pairs share a same sub-sequence of 5 nucleotides or more in length.

In an embodiment of the disclosure, each barcode is between 7 and 15 nucleotides in length, between 7 and 9 nucleotides in length, 8 nucleotides in length, between 10 and 12 nucleotides in length, or 11 nucleotides in length.

In an embodiment of the disclosure, the set consists of ninety-six unique barcodes. Optionally, each barcode of the set of barcodes consists of one of SEQ ID NOS: 9-104. Optionally, each barcode of the set of barcodes consisting of one of SEQ ID NOS: 105-200.

There is also provided in accordance with an embodiment of the disclosure a primer for use in a polymerase chain reaction, the primer comprising: a DNA barcode selected from a set of unique barcodes; and a direct repeat (DR) targeting region consisting of a DNA sequence complementary to portion of a DR of a CRISPR array. Optionally, the primer further comprises an adapter compatible with a Next Generation Sequencing system. Optionally, the barcode and the direct repeat targeting region are separated by no more than four intervening nucleotides. Optionally, the barcode and the direct repeat (DR) targeting region are directly adjacent to each other.

In an embodiment of the disclosure, the primer consists of the barcode and the DR targeting region arranged in a 5′ to 3′ direction, and ten or less additional nucleotides. Optionally, the barcode is preceded on its 5′ end with up to four nucleotides. Optionally, the primer consists of a guanine, the barcode and the DR targeting region arranged in a 5′ to 3′ direction. Optionally, the barcode consists of the adapter, the barcode and the DR targeting region arranged in a 5′ to 3′ direction, and ten or less additional nucleotides.

In an embodiment of the disclosure, the barcode is a barcode in accordance with any one of claims 1-7.

The is also provided in an embodiment of the disclosure, a method for characterizing spacer regions in a CRISPR array from each of a plurality of microbial DNA isolates, the method comprising: in a separate reaction well for each of the plurality of microbial DNA isolates, performing a PCR with a microbial DNA isolate and at least one pair of primers configured to amplify spacers within a CRISPR array comprised in the microbial DNA isolate and to add at least one barcode that uniquely indexes the PCR products produced in the reaction well, pooling the PCR products produced from each of the plurality of microbial DNA isolates; sequencing the pooled PCR products with a Next Generation Sequencing (NGS) system to obtain an aggregated sequence data; and computationally analyzing the aggregated sequence data to: demultiplex the aggregated sequence data to a plurality of bins based on the at least one barcode comprised in each PCR product, each bin comprising sequence data associated with one of the plurality of microbial DNA isolates; and for each bin, identify sequences encoding spacers.

In an embodiment of the disclosure, the at least one pair of primers comprises: a first primer pair configured to produce a first PCR product comprising at least one spacer amplified from the microbial DNA isolate and flanked on each side with a forward universal tail and a reverse universal tail, respectively; and a second primer pair configured to produce a second PCR product that amplifies the first PCR product and adds at least one barcode that uniquely indexes the second PCR product. Optionally, the first primer pair comprises: a forward primer comprising, in a 5′ to 3′ direction, an overhang region encoding a forward universal tail and a DR targeting region consisting of a DNA sequence complementary to portion of a DR of a CRISPR array; and a reverse primer comprising, in a 5′ to 3′ direction, an overhang region encoding a reverse universal tail and a DR targeting region consisting of a DNA sequence complementary to portion of a DR of a CRISPR array. Optionally, the second primer pair comprises: a forward primer comprising a targeting region complementary to at least a portion of the forward universal tail and a 5′ overhang comprising a barcode; and a reverse primer comprising a targeting region complementary to at least a portion of the reverse universal tail and a 5′ overhang comprising a barcode. Optionally, the 5′ overhang of the forward primer and the 5′ overhang of the reverse primer each further comprises an NGS adapter region for making the second PCR product compatible with an NGS system.

In an embodiment of the disclosure, the at least one pair of primers comprises a forward primer and a reverse primer, each primer comprising: a DR targeting region consisting of a DNA sequence complementary to portion of a DR of a CRISPR array; and a 5′ overhang region comprising a barcode. Optionally, the 5′ overhang of the forward primer and the 5′ overhang of the reverse primer each further comprises an NGS adapter region for making the PCR product compatible with an NGS system.

In an embodiment of the disclosure, each primer of the at least one pair of primers comprising a barcode comprises a same barcode, and each reaction well as well as the amplified DNA produced therein are characterized by a different barcode.

In an embodiment of the disclosure, each primer of the at least one pair of primers comprising a barcode comprises a different barcode, and each reaction well as well as the amplified DNA produced therein are characterized by a different combination of two barcodes.

In an embodiment of the disclosure, at least one PCR reaction of the plurality of PCR reactions is performed with at least one additional pair of primers configured to amplify a gene of interest or a portion thereof, each primer of the pair of primers comprising a targeting region consisting of a DNA sequence complementary to portion of the gene of interest. Optionally, the gene of interest is a gene associated with virulence, antibiotic resistance, or a lineage marker.

In an embodiment of the disclosure, identifying sequences encoding spacers comprises identifying within a set of demultiplexed sequence data a portion of the demultiplexed sequence data that is characterized by being flanked on both sides by a sequence encoding at least a portion of a DR of a CRISPR array comprised in the microbial DNA isolate.

In an embodiment of the disclosure, each reaction well is loaded with a plurality of pairs of primers configured to amplify spacers from a plurality of different CRISPR arrays comprised in the microbial DNA isolate. Optionally, the microbial DNA isolate is isolated from a biological sample comprising a plurality of different microbial species.

There is also provided in accordance with an embodiment of the disclosure, a method for performing a polymerase chain reaction (PCR) comprising: a first PCR for producing a first PCR product, the first PCR comprising: a template DNA; a first forward primer comprising, in a 5′ to 3′ direction, an overhang region encoding a forward universal tail and a targeting region consisting of a DNA sequence complementary to a portion of the template DNA; and a first reverse primer comprising, in a 5′ to 3′ direction, an overhang region encoding a reverse universal tail and a targeting region consisting of a DNA sequence complementary to a portion of the template DNA, wherein the first PCR product comprises at least one region amplified from the template DNA and is flanked on the ends with a forward universal tail and a reverse universal tail, respectively; and a second PCR for producing a second PCR reaction, the first PCR comprising: the first PCR product serving as a second template DNA; a second forward primer comprising a targeting region complementary to at least a portion of the forward universal tail; and a second reverse primer comprising a targeting region complementary to at least a portion of the reverse universal tail, wherein the second PCR product amplifies at least a portion of the first PCR product. Optionally, the forward primer and the reverse primer each comprise a 5′ overhang comprising a barcode for indexing the second PCR product. Optionally, the 5′ overhang of the forward primer and the 5′ overhang of the reverse primer each further comprises an NGS adapter region for making the second PCR product compatible with an NGS system. Optionally, the temperature of the annealing step for the first PCR is higher than the temperature of the annealing step for the second PCR. Optionally, the temperature of the annealing step for the first PCR is higher than the temperature of the annealing step for the second PCR by at least 8° C., or between 10° C. and 14° C.

There is also provided in accordance with an embodiment of the disclosure a phylogenetic tree, the method comprising: determining a profile of spacers respectively present in a CRISPR array comprised in each of a plurality of bacterial samples; calculating a distance respectively between each of a plurality of pairs of bacterial samples, each pair being a unique pair selected from the plurality of bacterial samples, wherein, for each unique pair, the distance is based on the count of spacers present in one sample but not in both samples of the unique pair; and generating a phylogenetic tree for the plurality of bacterial samples based on the respective distance between bacterial samples for each pair of the plurality of pairs of bacterial samples. Optionally, the phylogenetic tree is generated as a neighbor joining tree based on the respective distances determined for each of the pairs of bacterial samples.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the disclosure in the present application are provided by way of example and are not intended to limit the scope of the disclosure. The described embodiments comprise different features, not all of which are required in all embodiments. Some embodiments utilize only some of the features or possible combinations of the features. Variations of embodiments of the disclosure that are described, and embodiments comprising different combinations of features noted in the described embodiments, will occur to persons of the art. The scope of the invention is limited only by the claims.

1-16. (canceled)

17. A method for characterizing spacer regions in a CRISPR array from each of a plurality of microbial DNA isolates, the method comprising:
in a separate reaction well for each of the plurality of microbial DNA isolates, performing a PCR with a microbial DNA isolate and at least one pair of primers configured to amplify spacers within a CRISPR array comprised in the microbial DNA isolate and to add at least one barcode that uniquely indexes the PCR products produced in the reaction well,
pooling the PCR products produced from each of the plurality of microbial DNA isolates;
sequencing the pooled PCR products with a Next Generation Sequencing (NGS) system to obtain an aggregated sequence data; and
computationally analyzing the aggregated sequence data to:
demultiplex the aggregated sequence data to a plurality of bins based on the at least one barcode comprised in each PCR product, each bin comprising sequence data associated with one of the plurality of microbial DNA isolates; and
for each bin, identify sequences encoding spacers.

18. The method according to claim 17, wherein the at least one pair of primers comprises:
a first primer pair configured to produce a first PCR product comprising at least one spacer amplified from the microbial DNA isolate and flanked on each side with a forward universal tail and a reverse universal tail, respectively; and
a second primer pair configured to produce a second PCR product that amplifies the first PCR product and adds at least one barcode that uniquely indexes the second PCR product.

19. The method according to claim 18, wherein the first primer pair comprises:
a forward primer comprising, in a 5′ to 3′ direction, an overhang region encoding a forward universal tail and a DR targeting region consisting of a DNA sequence complementary to portion of a DR of a CRISPR array; and
a reverse primer comprising, in a 5′ to 3′ direction, an overhang region encoding a reverse universal tail and a DR targeting region consisting of a DNA sequence complementary to portion of a DR of a CRISPR array.

20. The method according to claim 18, wherein the second primer pair comprises:
a forward primer comprising a targeting region complementary to at least a portion of the forward universal tail and a 5′ overhang comprising a barcode; and
a reverse primer comprising a targeting region complementary to at least a portion of the reverse universal tail and a 5′ overhang comprising a barcode.

21. The method according to claim 20, wherein, in the second primer pair, the 5′ overhang of the forward primer and the 5′ overhang of the reverse primer each further comprises an NGS adapter region for making the second PCR product compatible with an NGS system.

22. The method according to claim 17, wherein the at least one pair of primers comprises a forward primer and a reverse primer, each primer comprising:
a DR targeting region consisting of a DNA sequence complementary to portion of a DR of a CRISPR array; and
a 5′ overhang region comprising a barcode.

23. The method according to claim 22, wherein the 5′ overhang of the forward primer and the 5′ overhang of the reverse primer each further comprises an NGS adapter region for making the PCR product compatible with an NGS system.

24. The method according to claim 17, each primer of the pair of primers comprising a barcode comprises a same barcode, and each reaction well as well as the amplified DNA produced therein are characterized by a different barcode.

25. The method according to claim 17, wherein each primer of the pair of primers comprising a barcode comprises a different barcode, and each reaction well as well as the amplified DNA produced therein are characterized by a different combination of two barcodes.

26. The method according to claim 17, wherein at least one PCR reaction of the plurality of PCR reactions is performed with at least one additional pair of primers configured to amplify a gene of interest or a portion thereof, each primer of the pair of primers comprising a targeting region consisting of a DNA sequence complementary to portion of the gene of interest.

27. The method according to claim 26, wherein the gene of interest is a gene associated with virulence, antibiotic resistance, or a lineage marker.

28. The method according to claim 17, wherein identifying sequences encoding spacers comprises identifying within a set of demultiplexed sequence data a portion of the demultiplexed sequence data that is characterized by being flanked on both sides by a sequence encoding at least a portion of a DR of a CRISPR array comprised in the microbial DNA isolate.

29. The method according to claim 17, wherein each reaction well is loaded with a plurality of pairs of primers configured to amplify spacers from a plurality of different CRISPR arrays comprised in the microbial DNA isolate.

30. The method according to claim 29, wherein the microbial DNA isolate is isolated from a biological sample comprising a plurality of different microbial species.

31. A method for performing a polymerase chain reaction (PCR) comprising:
a first PCR for producing a first PCR product, the first PCR comprising:
a template DNA;
a first forward primer comprising, in a 5′ to 3′ direction, an overhang region encoding a forward universal tail and a targeting region consisting of a DNA sequence complementary to a portion of the template DNA; and
a first reverse primer comprising, in a 5′ to 3′ direction, an overhang region encoding a reverse universal tail and a targeting region consisting of a DNA sequence complementary to a portion of the template DNA, wherein
the first PCR product comprises at least one region amplified from the template DNA and is flanked on the ends with a forward universal tail and a reverse universal tail, respectively; and
a second PCR for producing a second PCR reaction, the first PCR comprising:
the first PCR product serving as a second template DNA;
a second forward primer comprising a targeting region complementary to at least a portion of the forward universal tail; and
a second reverse primer comprising a targeting region complementary to at least a portion of the reverse universal tail, wherein
the second PCR product amplifies at least a portion of the first PCR product.

32. The method according to claim 31, wherein the second forward primer and the second reverse primer each comprise a 5′ overhang comprising a barcode for indexing the second PCR product.

33. The method according to claim 32, wherein the 5′ overhang of the second forward primer and the 5′ overhang of the second reverse primer each further comprises an NGS adapter region for making the second PCR product compatible with an NGS system.

34. The method according to claim 31, wherein the temperature of the annealing step for the first PCR is higher than the temperature of the annealing step for the second PCR.

35. The method according to claim 34, wherein the temperature of the annealing step for the first PCR is higher than the temperature of the annealing step for the second PCR by at least 8° C.

36. A method for generating a phylogenetic tree, the method comprising:
determining a profile of spacers respectively present in a CRISPR array comprised in each of a plurality of bacterial samples;
calculating a distance respectively between each of a plurality of pairs of bacterial samples, each pair being a unique pair selected from the plurality of bacterial samples, wherein, for each unique pair, the distance is based on the count of spacers present in one sample but not in both samples of the unique pair; and
generating a phylogenetic tree for the plurality of bacterial samples based on the respective distance between bacterial samples for each pair of the plurality of pairs of bacterial samples,
wherein the profile of spacers respectively present in a CRISPR array comprised in each of the plurality of bacterial samples is determined by the method according to claim 17.

37. (canceled)


SEQUENCE LISTING (265 sequences)

SEQ ID NO: 1 - DNA, 36 bp, Artificial
  DR of M.  tuberculosis
gtttccgtcccctctcggggttttgggtctgacgac

SEQ ID NO: 2 - DNA, 36 bp, Artificial
  Complementary segment of DR of SEQ ID NO 1
gtcgtcagacccaaaaccccgagaggggacggaaac

SEQ ID NO: 3 - DNA, 18 bp, Artificial
  DRa (DR1) Primer
ggttttgggtctgacgac

SEQ ID NO: 4 - DNA, 18 bp, Artificial
  DRb (DR2) Primer
ccgagaggggacggaaac

SEQ ID NO: 5 - DNA, 30 bp, Artificial
  DR1 HiCRISPR primer 201 (DR1-RL013-FOR)
gagactcgacgtggttttgggtctgacgac

SEQ ID NO: 6 - DNA, 11 bp, Artificial
  Barcode (204) of 11 bases within SEQ ID NO 5
agactcgacgt

SEQ ID NO: 7 - DNA, 30 bp, Artificial
  DR2-RL013-REV
gagactcgacgtccgagaggggacggaaac

SEQ ID NO: 8 - DNA, 11 bp, Artificial
  Barcode of 11 bases within DR2-RL013-REV
agactcgacgt

SEQ ID NO: 9 - DNA, 11 bp, Artificial
  11-base Barcodes
ggagttaggac

SEQ ID NO: 10 - DNA, 11 bp, Artificial
  11-base Barcodes
cctcaatcctg

SEQ ID NO: 11 - DNA, 11 bp, Artificial
  11-base Barcodes
ttctggcttca

SEQ ID NO: 12 - DNA, 11 bp, Artificial
  11-base Barcodes
aagaccgaagt

SEQ ID NO: 13 - DNA, 11 bp, Artificial
  11-base Barcodes
tgtccacgttg

SEQ ID NO: 14 - DNA, 11 bp, Artificial
  11-base Barcodes
acaggtgcaac

SEQ ID NO: 15 - DNA, 11 bp, Artificial
  11-base Barcodes
gtgaacatggt

SEQ ID NO: 16 - DNA, 11 bp, Artificial
  11-base Barcodes
cacttgtacca

SEQ ID NO: 17 - DNA, 11 bp, Artificial
  11-base Barcodes
tgcagcatatt

SEQ ID NO: 18 - DNA, 11 bp, Artificial
  11-base Barcodes
acgtcgtataa

SEQ ID NO: 19 - DNA, 11 bp, Artificial
  11-base Barcodes
gtaatacgcgg

SEQ ID NO: 20 - DNA, 11 bp, Artificial
  11-base Barcodes
ttactgtaggt

SEQ ID NO: 21 - DNA, 11 bp, Artificial
  11-base Barcodes
aatgacatcca

SEQ ID NO: 22 - DNA, 11 bp, Artificial
  11-base Barcodes
ggcagttcttg

SEQ ID NO: 23 - DNA, 11 bp, Artificial
  11-base Barcodes
cagtcacgaac

SEQ ID NO: 24 - DNA, 11 bp, Artificial
  11-base Barcodes
cgtaaccgcat

SEQ ID NO: 25 - DNA, 11 bp, Artificial
  11-base Barcodes
gcattggcgta

SEQ ID NO: 26 - DNA, 11 bp, Artificial
  11-base Barcodes
tacggttatgc

SEQ ID NO: 27 - DNA, 11 bp, Artificial
  11-base Barcodes
ggttccaatta

SEQ ID NO: 28 - DNA, 11 bp, Artificial
  11-base Barcodes
ttggaaccagc

SEQ ID NO: 29 - DNA, 11 bp, Artificial
  11-base Barcodes
ctctccttagc

SEQ ID NO: 30 - DNA, 11 bp, Artificial
  11-base Barcodes
gagaggaatcg

SEQ ID NO: 31 - DNA, 11 bp, Artificial
  11-base Barcodes
agagaaggcta

SEQ ID NO: 32 - DNA, 11 bp, Artificial
  11-base Barcodes
tctcttccgat

SEQ ID NO: 33 - DNA, 11 bp, Artificial
  11-base Barcodes
ggatgtcaagg

SEQ ID NO: 34 - DNA, 11 bp, Artificial
  11-base Barcodes
ttcgtgacctt

SEQ ID NO: 35 - DNA, 11 bp, Artificial
  11-base Barcodes
ctcggattgtg

SEQ ID NO: 36 - DNA, 11 bp, Artificial
  11-base Barcodes
gagcctaacac

SEQ ID NO: 37 - DNA, 11 bp, Artificial
  11-base Barcodes
agattcggtgt

SEQ ID NO: 38 - DNA, 11 bp, Artificial
  11-base Barcodes
tctaagccaca

SEQ ID NO: 39 - DNA, 11 bp, Artificial
  11-base Barcodes
tgaatagacga

SEQ ID NO: 40 - DNA, 11 bp, Artificial
  11-base Barcodes
acttatctgct

SEQ ID NO: 41 - DNA, 11 bp, Artificial
  11-base Barcodes
gtccgctcata

SEQ ID NO: 42 - DNA, 11 bp, Artificial
  11-base Barcodes
actgcctgtgt

SEQ ID NO: 43 - DNA, 11 bp, Artificial
  11-base Barcodes
tgacggacaca

SEQ ID NO: 44 - DNA, 11 bp, Artificial
  11-base Barcodes
cagtaagtgtg

SEQ ID NO: 45 - DNA, 11 bp, Artificial
  11-base Barcodes
gtcattcacac

SEQ ID NO: 46 - DNA, 11 bp, Artificial
  11-base Barcodes
agcagactctg

SEQ ID NO: 47 - DNA, 11 bp, Artificial
  11-base Barcodes
tcgtctgagac

SEQ ID NO: 48 - DNA, 11 bp, Artificial
  11-base Barcodes
ctactcagagt

SEQ ID NO: 49 - DNA, 11 bp, Artificial
  11-base Barcodes
gatgagtctca

SEQ ID NO: 50 - DNA, 11 bp, Artificial
  11-base Barcodes
caccaagtcga

SEQ ID NO: 51 - DNA, 11 bp, Artificial
  11-base Barcodes
acaacctgatc

SEQ ID NO: 52 - DNA, 11 bp, Artificial
  11-base Barcodes
ttgctagtgga

SEQ ID NO: 53 - DNA, 11 bp, Artificial
  11-base Barcodes
aacgatcacct

SEQ ID NO: 54 - DNA, 11 bp, Artificial
  11-base Barcodes
ccatcgacaag

SEQ ID NO: 55 - DNA, 11 bp, Artificial
  11-base Barcodes
tgtgcggtaga

SEQ ID NO: 56 - DNA, 11 bp, Artificial
  11-base Barcodes
acgccaactag

SEQ ID NO: 57 - DNA, 11 bp, Artificial
  11-base Barcodes
tcatgcgcgat

SEQ ID NO: 58 - DNA, 11 bp, Artificial
  11-base Barcodes
tcatgcgcgat

SEQ ID NO: 59 - DNA, 11 bp, Artificial
  11-base Barcodes
tcatgcgcgat

SEQ ID NO: 60 - DNA, 11 bp, Artificial
  11-base Barcodes
tcatgcgcgat

SEQ ID NO: 61 - DNA, 11 bp, Artificial
  11-base Barcodes
tcatgcgcgat

SEQ ID NO: 62 - DNA, 11 bp, Artificial
  11-base Barcodes
tcatgcgcgat

SEQ ID NO: 63 - DNA, 11 bp, Artificial
  11-base Barcodes
tcatgcgcgat

SEQ ID NO: 64 - DNA, 11 bp, Artificial
  11-base Barcodes
tcatgcgcgat

SEQ ID NO: 65 - DNA, 11 bp, Artificial
  11-base Barcodes
tcatgcgcgat

SEQ ID NO: 66 - DNA, 11 bp, Artificial
  11-base Barcodes
tcatgcgcgat

SEQ ID NO: 67 - DNA, 11 bp, Artificial
  11-base Barcodes
tcatgcgcgat

SEQ ID NO: 68 - DNA, 11 bp, Artificial
  11-base Barcodes
tcatgcgcgat

SEQ ID NO: 69 - DNA, 11 bp, Artificial
  11-base Barcodes
tcatgcgcgat

SEQ ID NO: 70 - DNA, 11 bp, Artificial
  11-base Barcodes
tcatgcgcgat

SEQ ID NO: 71 - DNA, 11 bp, Artificial
  11-base Barcodes
tcatgcgcgat

SEQ ID NO: 72 - DNA, 11 bp, Artificial
  11-base Barcodes
tcatgcgcgat

SEQ ID NO: 73 - DNA, 11 bp, Artificial
  11-base Barcodes
tcatgcgcgat

SEQ ID NO: 74 - DNA, 11 bp, Artificial
  11-base Barcodes
tcatgcgcgat

SEQ ID NO: 75 - DNA, 11 bp, Artificial
  11-base Barcodes
tcatgcgcgat

SEQ ID NO: 76 - DNA, 11 bp, Artificial
  11-base Barcodes
tcatgcgcgat

SEQ ID NO: 77 - DNA, 11 bp, Artificial
  11-base Barcodes
tcatgcgcgat

SEQ ID NO: 78 - DNA, 11 bp, Artificial
  11-base Barcodes
tcatgcgcgat

SEQ ID NO: 79 - DNA, 11 bp, Artificial
  11-base Barcodes
tcatgcgcgat

SEQ ID NO: 80 - DNA, 11 bp, Artificial
  11-base Barcodes
tcatgcgcgat

SEQ ID NO: 81 - DNA, 11 bp, Artificial
  11-base Barcodes
tcatgcgcgat

SEQ ID NO: 82 - DNA, 11 bp, Artificial
  11-base Barcodes
tcatgcgcgat

SEQ ID NO: 83 - DNA, 11 bp, Artificial
  11-base Barcodes
tcatgcgcgat

SEQ ID NO: 84 - DNA, 11 bp, Artificial
  11-base Barcodes
tcatgcgcgat

SEQ ID NO: 85 - DNA, 11 bp, Artificial
  11-base Barcodes
tcatgcgcgat

SEQ ID NO: 86 - DNA, 11 bp, Artificial
  11-base Barcodes
tcatgcgcgat

SEQ ID NO: 87 - DNA, 11 bp, Artificial
  11-base Barcodes
tcatgcgcgat

SEQ ID NO: 88 - DNA, 11 bp, Artificial
  11-base Barcodes
tcatgcgcgat

SEQ ID NO: 89 - DNA, 11 bp, Artificial
  11-base Barcodes
tcatgcgcgat

SEQ ID NO: 90 - DNA, 11 bp, Artificial
  11-base Barcodes
tcatgcgcgat

SEQ ID NO: 91 - DNA, 11 bp, Artificial
  11-base Barcodes
tcatgcgcgat

SEQ ID NO: 92 - DNA, 11 bp, Artificial
  11-base Barcodes
tcatgcgcgat

SEQ ID NO: 93 - DNA, 11 bp, Artificial
  11-base Barcodes
tcatgcgcgat

SEQ ID NO: 94 - DNA, 11 bp, Artificial
  11-base Barcodes
tcatgcgcgat

SEQ ID NO: 95 - DNA, 11 bp, Artificial
  11-base Barcodes
tcatgcgcgat

SEQ ID NO: 96 - DNA, 11 bp, Artificial
  11-base Barcodes
tcatgcgcgat

SEQ ID NO: 97 - DNA, 11 bp, Artificial
  11-base Barcodes
tcatgcgcgat

SEQ ID NO: 98 - DNA, 11 bp, Artificial
  11-base Barcodes
tcatgcgcgat

SEQ ID NO: 99 - DNA, 11 bp, Artificial
  11-base Barcodes
tcatgcgcgat

SEQ ID NO: 100 - DNA, 11 bp, Artificial
  11-base Barcodes
tcatgcgcgat

SEQ ID NO: 101 - DNA, 11 bp, Artificial
  11-base Barcodes
tcatgcgcgat

SEQ ID NO: 102 - DNA, 11 bp, Artificial
  11-base Barcodes
tcatgcgcgat

SEQ ID NO: 103 - DNA, 11 bp, Artificial
  11-base Barcodes
tcatgcgcgat

SEQ ID NO: 104 - DNA, 11 bp, Artificial
  11-base Barcodes
tcatgcgcgat

SEQ ID NO: 105 - DNA, 8 bp, Artificial
  8-base barcodes
aagcagga

SEQ ID NO: 106 - DNA, 8 bp, Artificial
  8-base barcodes
ttcgtcct

SEQ ID NO: 107 - DNA, 8 bp, Artificial
  8-base barcodes
cctacttc

SEQ ID NO: 108 - DNA, 8 bp, Artificial
  8-base barcodes
ggatgaag

SEQ ID NO: 109 - DNA, 8 bp, Artificial
  8-base barcodes
aacagcaa

SEQ ID NO: 110 - DNA, 8 bp, Artificial
  8-base barcodes
ttgtcgtt

SEQ ID NO: 111 - DNA, 8 bp, Artificial
  8-base barcodes
gttgatgg

SEQ ID NO: 112 - DNA, 8 bp, Artificial
  8-base barcodes
agagtgtt

SEQ ID NO: 113 - DNA, 8 bp, Artificial
  8-base barcodes
tctcacaa

SEQ ID NO: 114 - DNA, 8 bp, Artificial
  8-base barcodes
aagacacc

SEQ ID NO: 115 - DNA, 8 bp, Artificial
  8-base barcodes
caggtagt

SEQ ID NO: 116 - DNA, 8 bp, Artificial
  8-base barcodes
acttgctg

SEQ ID NO: 117 - DNA, 8 bp, Artificial
  8-base barcodes
tgaacgac

SEQ ID NO: 118 - DNA, 8 bp, Artificial
  8-base barcodes
gtccagat

SEQ ID NO: 119 - DNA, 8 bp, Artificial
  8-base barcodes
cgctatac

SEQ ID NO: 120 - DNA, 8 bp, Artificial
  8-base barcodes
gcgatatg

SEQ ID NO: 121 - DNA, 8 bp, Artificial
  8-base barcodes
gtcgcata

SEQ ID NO: 122 - DNA, 8 bp, Artificial
  8-base barcodes
ggtatcat

SEQ ID NO: 123 - DNA, 8 bp, Artificial
  8-base barcodes
ccatagta

SEQ ID NO: 124 - DNA, 8 bp, Artificial
  8-base barcodes
ctaccact

SEQ ID NO: 125 - DNA, 8 bp, Artificial
  8-base barcodes
aatgcgag

SEQ ID NO: 126 - DNA, 8 bp, Artificial
  8-base barcodes
ttacgctc

SEQ ID NO: 127 - DNA, 8 bp, Artificial
  8-base barcodes
gaaggtct

SEQ ID NO: 128 - DNA, 8 bp, Artificial
  8-base barcodes
tccttgag

SEQ ID NO: 129 - DNA, 8 bp, Artificial
  8-base barcodes
gtgagtac

SEQ ID NO: 130 - DNA, 8 bp, Artificial
  8-base barcodes
tatcgagg

SEQ ID NO: 131 - DNA, 8 bp, Artificial
  8-base barcodes
agcattcg

SEQ ID NO: 132 - DNA, 8 bp, Artificial
  8-base barcodes
tcgtaagc

SEQ ID NO: 133 - DNA, 8 bp, Artificial
  8-base barcodes
tgtgctca

SEQ ID NO: 134 - DNA, 8 bp, Artificial
  8-base barcodes
cataaccg

SEQ ID NO: 135 - DNA, 8 bp, Artificial
  8-base barcodes
gtattggc

SEQ ID NO: 136 - DNA, 8 bp, Artificial
  8-base barcodes
tctaggct

SEQ ID NO: 137 - DNA, 8 bp, Artificial
  8-base barcodes
agatccga

SEQ ID NO: 138 - DNA, 8 bp, Artificial
  8-base barcodes
gagttcca

SEQ ID NO: 139 - DNA, 8 bp, Artificial
  8-base barcodes
tacgagtc

SEQ ID NO: 140 - DNA, 8 bp, Artificial
  8-base barcodes
actcttgt

SEQ ID NO: 141 - DNA, 8 bp, Artificial
  8-base barcodes
agtcaatc

SEQ ID NO: 142 - DNA, 8 bp, Artificial
  8-base barcodes
cttctgtg

SEQ ID NO: 143 - DNA, 8 bp, Artificial
  8-base barcodes
tacactgt

SEQ ID NO: 144 - DNA, 8 bp, Artificial
  8-base barcodes
tatgtacc

SEQ ID NO: 145 - DNA, 8 bp, Artificial
  8-base barcodes
ccgaatga

SEQ ID NO: 146 - DNA, 8 bp, Artificial
  8-base barcodes
ggcttact

SEQ ID NO: 147 - DNA, 8 bp, Artificial
  8-base barcodes
ccagttac

SEQ ID NO: 148 - DNA, 8 bp, Artificial
  8-base barcodes
gccaactt

SEQ ID NO: 149 - DNA, 8 bp, Artificial
  8-base barcodes
aacctgac

SEQ ID NO: 150 - DNA, 8 bp, Artificial
  8-base barcodes
ttggactg

SEQ ID NO: 151 - DNA, 8 bp, Artificial
  8-base barcodes
ataggagc

SEQ ID NO: 152 - DNA, 8 bp, Artificial
  8-base barcodes
cgatgtca

SEQ ID NO: 153 - DNA, 8 bp, Artificial
  8-base barcodes
gctacagt

SEQ ID NO: 154 - DNA, 8 bp, Artificial
  8-base barcodes
acgtggaa

SEQ ID NO: 155 - DNA, 8 bp, Artificial
  8-base barcodes
atggatct

SEQ ID NO: 156 - DNA, 8 bp, Artificial
  8-base barcodes
atggtcac

SEQ ID NO: 157 - DNA, 8 bp, Artificial
  8-base barcodes
caccaatt

SEQ ID NO: 158 - DNA, 8 bp, Artificial
  8-base barcodes
ggacaaca

SEQ ID NO: 159 - DNA, 8 bp, Artificial
  8-base barcodes
ttgcgtga

SEQ ID NO: 160 - DNA, 8 bp, Artificial
  8-base barcodes
ggttggtt

SEQ ID NO: 161 - DNA, 8 bp, Artificial
  8-base barcodes
cgtgtcta

SEQ ID NO: 162 - DNA, 8 bp, Artificial
  8-base barcodes
cattgtgt

SEQ ID NO: 163 - DNA, 8 bp, Artificial
  8-base barcodes
gactcaac

SEQ ID NO: 164 - DNA, 8 bp, Artificial
  8-base barcodes
tgctaatg

SEQ ID NO: 165 - DNA, 8 bp, Artificial
  8-base barcodes
gtagccat

SEQ ID NO: 166 - DNA, 8 bp, Artificial
  8-base barcodes
agcgcaat

SEQ ID NO: 167 - DNA, 8 bp, Artificial
  8-base barcodes
gataccta

SEQ ID NO: 168 - DNA, 8 bp, Artificial
  8-base barcodes
ctctgaat

SEQ ID NO: 169 - DNA, 8 bp, Artificial
  8-base barcodes
aggaagat

SEQ ID NO: 170 - DNA, 8 bp, Artificial
  8-base barcodes
accgtaga

SEQ ID NO: 171 - DNA, 8 bp, Artificial
  8-base barcodes
taactggt

SEQ ID NO: 172 - DNA, 8 bp, Artificial
  8-base barcodes
aactacgg

SEQ ID NO: 173 - DNA, 8 bp, Artificial
  8-base barcodes
cacatgct

SEQ ID NO: 174 - DNA, 8 bp, Artificial
  8-base barcodes
ctgtaaca

SEQ ID NO: 175 - DNA, 8 bp, Artificial
  8-base barcodes
gagcctat

SEQ ID NO: 176 - DNA, 8 bp, Artificial
  8-base barcodes
cgtcatct

SEQ ID NO: 177 - DNA, 8 bp, Artificial
  8-base barcodes
actgagca

SEQ ID NO: 178 - DNA, 8 bp, Artificial
  8-base barcodes
ttagagga

SEQ ID NO: 179 - DNA, 8 bp, Artificial
  8-base barcodes
cctagaag

SEQ ID NO: 180 - DNA, 8 bp, Artificial
  8-base barcodes
atactccg

SEQ ID NO: 181 - DNA, 8 bp, Artificial
  8-base barcodes
gcgtctta

SEQ ID NO: 182 - DNA, 8 bp, Artificial
  8-base barcodes
cagagata

SEQ ID NO: 183 - DNA, 8 bp, Artificial
  8-base barcodes
tcagaact

SEQ ID NO: 184 - DNA, 8 bp, Artificial
  8-base barcodes
ttcacgca

SEQ ID NO: 185 - DNA, 8 bp, Artificial
  8-base barcodes
atcaagtg

SEQ ID NO: 186 - DNA, 8 bp, Artificial
  8-base barcodes
tagtgcac

SEQ ID NO: 187 - DNA, 8 bp, Artificial
  8-base barcodes
gttcttcc

SEQ ID NO: 188 - DNA, 8 bp, Artificial
  8-base barcodes
ccttcgat

SEQ ID NO: 189 - DNA, 8 bp, Artificial
  8-base barcodes
ccattagg

SEQ ID NO: 190 - DNA, 8 bp, Artificial
  8-base barcodes
taggttag

SEQ ID NO: 191 - DNA, 8 bp, Artificial
  8-base barcodes
gcacgatt

SEQ ID NO: 192 - DNA, 8 bp, Artificial
  8-base barcodes
caagctga

SEQ ID NO: 193 - DNA, 8 bp, Artificial
  8-base barcodes
tctgcctt

SEQ ID NO: 194 - DNA, 8 bp, Artificial
  8-base barcodes
acatatcc

SEQ ID NO: 195 - DNA, 8 bp, Artificial
  8-base barcodes
cttaaggc

SEQ ID NO: 196 - DNA, 8 bp, Artificial
  8-base barcodes
tagcacct

SEQ ID NO: 197 - DNA, 8 bp, Artificial
  8-base barcodes
ggaattga

SEQ ID NO: 198 - DNA, 8 bp, Artificial
  8-base barcodes
taatcgcg

SEQ ID NO: 199 - DNA, 8 bp, Artificial
  8-base barcodes
gaagtctc

SEQ ID NO: 200 - DNA, 8 bp, Artificial
  8-base barcodes
aagcgttc

SEQ ID NO: 201 - DNA, 51 bp, Artificial
  UT-DRa1
tcgtcggcagcgtcagatgtgtataagagacagggttttgggtctgacgac

SEQ ID NO: 202 - DNA, 52 bp, Artificial
  UT-DRa2
  misc_feature (34)..(34): n is a, c, g, or t
tcgtcggcagcgtcagatgtgtataagagacagnggttttgggtctgacgac

SEQ ID NO: 203 - DNA, 53 bp, Artificial
  UT-DRa3
  misc_feature (34)..(35): n is a, c, g, or t
tcgtcggcagcgtcagatgtgtataagagacagnnggttttgggtctgacgac

SEQ ID NO: 204 - DNA, 54 bp, Artificial
  UT-DRa4
  misc_feature (34)..(36): n is a, c, g, or t
tcgtcggcagcgtcagatgtgtataagagacagnnnggttttgggtctgacgac

SEQ ID NO: 205 - DNA, 52 bp, Artificial
  UT-DRb1
gtctcgtgggctcggagatgtgtataagagacagccgagaggggacggaaac

SEQ ID NO: 206 - DNA, 53 bp, Artificial
  UT-DRb2
  misc_feature (35)..(35): n is a, c, g, or t
gtctcgtgggctcggagatgtgtataagagacagnccgagaggggacggaaac

SEQ ID NO: 207 - DNA, 54 bp, Artificial
  UT-DRb3
  misc_feature (35)..(36): n is a, c, g, or t
gtctcgtgggctcggagatgtgtataagagacagnnccgagaggggacggaaac

SEQ ID NO: 208 - DNA, 55 bp, Artificial
  UT-DRb4
  misc_feature (35)..(37): n is a, c, g, or t
gtctcgtgggctcggagatgtgtataagagacagnnnccgagaggggacggaaac

SEQ ID NO: 209 - DNA, 14 bp, Artificial
  Forward universal tail overhang
tcgtcggcagcgtc

SEQ ID NO: 210 - DNA, 19 bp, Artificial
  Mosaic End overhang
agatgtgtataagagacag

SEQ ID NO: 211 - DNA, 15 bp, Artificial
  Reverse universal tail overhang
gtctcgtgggctcgg

SEQ ID NO: 212 - DNA, 54 bp, Artificial
  proMID-1F
aatgatacggcgaccaccgagatctacacggagttaggactcgtcggcagcgtc

SEQ ID NO: 213 - DNA, 54 bp, Artificial
  proMID-2F
aatgatacggcgaccaccgagatctacaccctcaatcctgtcgtcggcagcgtc

SEQ ID NO: 214 - DNA, 54 bp, Artificial
  proMID-3F
aatgatacggcgaccaccgagatctacacttctggcttcatcgtcggcagcgtc

SEQ ID NO: 215 - DNA, 54 bp, Artificial
  proMID-4F
aatgatacggcgaccaccgagatctacacaagaccgaagttcgtcggcagcgtc

SEQ ID NO: 216 - DNA, 54 bp, Artificial
  proMID-5F
aatgatacggcgaccaccgagatctacactgtccacgttgtcgtcggcagcgtc

SEQ ID NO: 217 - DNA, 54 bp, Artificial
  proMID-6F
aatgatacggcgaccaccgagatctacacacaggtgcaactcgtcggcagcgtc

SEQ ID NO: 218 - DNA, 54 bp, Artificial
  proMID-7F
aatgatacggcgaccaccgagatctacacgtgaacatggttcgtcggcagcgtc

SEQ ID NO: 219 - DNA, 54 bp, Artificial
  proMID-8F
aatgatacggcgaccaccgagatctacaccacttgtaccatcgtcggcagcgtc

SEQ ID NO: 220 - DNA, 54 bp, Artificial
  proMID-9F
aatgatacggcgaccaccgagatctacactgcagcatatttcgtcggcagcgtc

SEQ ID NO: 221 - DNA, 54 bp, Artificial
  proMID-10F
aatgatacggcgaccaccgagatctacacacgtcgtataatcgtcggcagcgtc

SEQ ID NO: 222 - DNA, 54 bp, Artificial
  proMID-11F
aatgatacggcgaccaccgagatctacacgtaatacgcggtcgtcggcagcgtc

SEQ ID NO: 223 - DNA, 54 bp, Artificial
  proMID-12F
aatgatacggcgaccaccgagatctacacttactgtaggttcgtcggcagcgtc

SEQ ID NO: 224 - DNA, 50 bp, Artificial
  proMID-13R
caagcagaagacggcatacgagataatgacatccagtctcgtgggctcgg

SEQ ID NO: 225 - DNA, 50 bp, Artificial
  proMID-14R
caagcagaagacggcatacgagatggcagttcttggtctcgtgggctcgg

SEQ ID NO: 226 - DNA, 50 bp, Artificial
  proMID-15R
caagcagaagacggcatacgagatcagtcacgaacgtctcgtgggctcgg

SEQ ID NO: 227 - DNA, 50 bp, Artificial
  proMID-16R
caagcagaagacggcatacgagatcgtaaccgcatgtctcgtgggctcgg

SEQ ID NO: 228 - DNA, 50 bp, Artificial
  proMID-17R
caagcagaagacggcatacgagatgcattggcgtagtctcgtgggctcgg

SEQ ID NO: 229 - DNA, 50 bp, Artificial
  proMID-18R
caagcagaagacggcatacgagattacggttatgcgtctcgtgggctcgg

SEQ ID NO: 230 - DNA, 50 bp, Artificial
  proMID-19R
caagcagaagacggcatacgagatggttccaattagtctcgtgggctcgg

SEQ ID NO: 231 - DNA, 50 bp, Artificial
  proMID-20R
caagcagaagacggcatacgagatttggaaccagcgtctcgtgggctcgg

SEQ ID NO: 232 - DNA, 29 bp, Artificial
  P5 universal adapter overhang
aatgatacggcgaccaccgagatctacac

SEQ ID NO: 233 - DNA, 24 bp, Artificial
  P7 universal adapter overhang
caagcagaagacggcatacgagat

SEQ ID NO: 234 - DNA, 19 bp, Artificial
  mDRa
gggttttgggtctgacgac

SEQ ID NO: 235 - DNA, 17 bp, Artificial
  mDRb
cgagaggggacggaaac

SEQ ID NO: 236 - DNA, 11 bp, Artificial
  sUT-F
tcgtcggcagc

SEQ ID NO: 237 - DNA, 12 bp, Artificial
  sUT-R
gtctcgtgggct

SEQ ID NO: 238 - DNA, 52 bp, Artificial
  UT-mDRa1
tcgtcggcagcgtcagatgtgtataagagacaggggttttgggtctgacgac

SEQ ID NO: 239 - DNA, 53 bp, Artificial
  UT-mDRa2
  misc_feature (34)..(34): n is a, c, g, or t
tcgtcggcagcgtcagatgtgtataagagacagngggttttgggtctgacgac

SEQ ID NO: 240 - DNA, 54 bp, Artificial
  UT-mDRa3
  misc_feature (34)..(35): n is a, c, g, or t
tcgtcggcagcgtcagatgtgtataagagacagnngggttttgggtctgacgac

SEQ ID NO: 241 - DNA, 55 bp, Artificial
  UT-mDRa4
  misc_feature (34)..(36): n is a, c, g, or t
tcgtcggcagcgtcagatgtgtataagagacagnnngggttttgggtctgacgac

SEQ ID NO: 242 - DNA, 51 bp, Artificial
  UT-mDRb1
gtctcgtgggctcggagatgtgtataagagacagcgagaggggacggaaac

SEQ ID NO: 243 - DNA, 52 bp, Artificial
  UT-mDRb2
  misc_feature (35)..(35): n is a, c, g, or t
gtctcgtgggctcggagatgtgtataagagacagncgagaggggacggaaac

SEQ ID NO: 244 - DNA, 53 bp, Artificial
  UT-mDRb3
  misc_feature (35)..(36): n is a, c, g, or t
gtctcgtgggctcggagatgtgtataagagacagnncgagaggggacggaaac

SEQ ID NO: 245 - DNA, 54 bp, Artificial
  UT-mDRb4
  misc_feature (35)..(37): n is a, c, g, or t
gtctcgtgggctcggagatgtgtataagagacagnnncgagaggggacggaaac

SEQ ID NO: 246 - DNA, 48 bp, Artificial
  1Fnew
aatgatacggcgaccaccgagatctacacaagcaggatcgtcggcagc

SEQ ID NO: 247 - DNA, 48 bp, Artificial
  2Fnew
aatgatacggcgaccaccgagatctacacttcgtccttcgtcggcagc

SEQ ID NO: 248 - DNA, 48 bp, Artificial
  3Fnew
aatgatacggcgaccaccgagatctacaccctacttctcgtcggcagc

SEQ ID NO: 249 - DNA, 48 bp, Artificial
  4Fnew
aatgatacggcgaccaccgagatctacacggatgaagtcgtcggcagc

SEQ ID NO: 250 - DNA, 48 bp, Artificial
  5Fnew
aatgatacggcgaccaccgagatctacacaacagcaatcgtcggcagc

SEQ ID NO: 251 - DNA, 48 bp, Artificial
  6Fnew
aatgatacggcgaccaccgagatctacacttgtcgtttcgtcggcagc

SEQ ID NO: 252 - DNA, 48 bp, Artificial
  7Fnew
aatgatacggcgaccaccgagatctacacgttgatggtcgtcggcagc

SEQ ID NO: 253 - DNA, 48 bp, Artificial
  8Fnew
aatgatacggcgaccaccgagatctacacagagtgtttcgtcggcagc

SEQ ID NO: 254 - DNA, 48 bp, Artificial
  9Fnew
aatgatacggcgaccaccgagatctacactctcacaatcgtcggcagc

SEQ ID NO: 255 - DNA, 48 bp, Artificial
  10Fnew
aatgatacggcgaccaccgagatctacacaagacacctcgtcggcagc

SEQ ID NO: 256 - DNA, 48 bp, Artificial
  11Fnew
aatgatacggcgaccaccgagatctacaccaggtagttcgtcggcagc

SEQ ID NO: 257 - DNA, 48 bp, Artificial
  12Fnew
aatgatacggcgaccaccgagatctacacacttgctgtcgtcggcagc

SEQ ID NO: 258 - DNA, 44 bp, Artificial
  13Rev_new
caagcagaagacggcatacgagatgtcgttcagtctcgtgggct

SEQ ID NO: 259 - DNA, 44 bp, Artificial
  14Rev_new
caagcagaagacggcatacgagatatctggacgtctcgtgggct

SEQ ID NO: 260 - DNA, 44 bp, Artificial
  15Rev_new
caagcagaagacggcatacgagatgtatagcggtctcgtgggct

SEQ ID NO: 261 - DNA, 44 bp, Artificial
  16Rev_new
caagcagaagacggcatacgagatcatatcgcgtctcgtgggct

SEQ ID NO: 262 - DNA, 44 bp, Artificial
  17Rev_new
caagcagaagacggcatacgagattatgcgacgtctcgtgggct

SEQ ID NO: 263 - DNA, 44 bp, Artificial
  18Rev_new
caagcagaagacggcatacgagatatgataccgtctcgtgggct

SEQ ID NO: 264 - DNA, 44 bp, Artificial
  19Rev_new
caagcagaagacggcatacgagattactatgggtctcgtgggct

SEQ ID NO: 265 - DNA, 44 bp, Artificial
  20Rev_new
caagcagaagacggcatacgagatagtggtaggtctcgtgggct